(19) United States
(12) Patent Application Publication
(10) Pub. No.: US 2025/0327018 A1
(43) Pub. Date: Oct. 23, 2025

(54) RARE EARTH METAL EXTRACTING BACTERIAL CONSORTIA

(71) Applicants: Miles Rogers, Watertown, MA (US); Samy Ashkar, Portsmith, RI (US)

(72) Inventors: Miles Rogers, Watertown, MA (US); Samy Ashkar, Portsmith, RI (US)

(21) Appl. No.: 19/171,043

(22) Filed: Apr. 4, 2025

Related U.S. Application Data
(63) Continuation of application No. 18/110,325, filed on Feb. 15, 2023, now abandoned.
(60) Provisional application No. 63/311,005, filed on Feb. 16, 2022.
(60) Provisional application No. 63/311,012, filed on Feb. 16, 2022.

(51) Int. Cl. C12N 1/20; C01F 17/10; C01F 17/224; C01F 17/276; C01F 17/282; C01G 49/00

(52) U.S. Cl. CPC C12N 1/20; C01F 17/10; C01F 17/224; C01F 17/276; C01F 17/282; C01G 49/00

(57) ABSTRACT

A rare earth metal extracting bacterial consortium can include an acid secreting bacterium, a heavy metal resistant bacterium, an iron-sequestering molecule secreting bacterium, and a rare earth metal sequestering bacterium. In another example, a composition can include a growth medium and a bacterial consortium growing in the growth medium. The growth medium can include water, magnesium sulfate, manganese chloride, cobalt chloride, calcium chloride, ammonium sulfate, soluble starch, and amino acids. The bacterial consortium can include an acid secreting bacterium, a heavy metal resistant bacterium, an iron-sequestering molecule secreting bacterium, and a rare earth metal sequestering bacterium.

### RELATED APPLICATIONS

This application is a continuation of U.S. patent application Ser. No. 18/110,325, filed Feb. 15, 2023, which claims the benefit of U.S. Provisional Patent Application Ser. No. 63/311,005 and U.S. Provisional Patent Application Ser. No. 63/311,012, each of which was filed on Feb. 16, 2022, which are all incorporated herein by reference in their entirety. This application also incorporates by reference the subject matter of published United States Patent Application 2023/0257278, filed on Feb. 15, 2023.

### BACKGROUND

Rare earth metals, also called rare earth elements, include the lanthanides and often yttrium and scandium. These metals have unique properties that make them useful in many applications, including electronics, magnets, lasers, glass, alloys, and others. As an example, “rare earth magnets” are magnets made up of an alloy including neodymium, iron, and boron. Rare earth magnets include some of the strongest permanent magnets currently available. Rare earth metals tend to be found in low concentrations dispersed in other ores or minerals. Therefore, obtaining a useful amount or concentration of rare earth metals usually requires separating, purifying, etc. a large volume of raw material and can be expensive. Additionally, many rare earth metals are used in electronic devices that have a limited operating life span, after which the devices are discarded or recycled. Recycling the rare earth metals in these devices is an attractive possibility. However, many recycling processes for rare earth metals are expensive and/or harmful to the environment.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIG. 1 is a schematic representation of an example rare earth metal extracting bacterial consortium in accordance with an example of the present invention.

FIG. 2 is a schematic representation of a composition including a growth medium and a rare earth metal extracting bacterial consortium in accordance with another example of the present invention.

FIG. 3 is a schematic diagram of a metabolic pathway used by Bacillus sp. to produce citric acid in accordance with still another example of the present invention.

FIG. 4 is a schematic diagram of a metabolic pathway used by Butyrivibrio hungatei to produce butyric acid in accordance with an example of the present invention.

FIG. 5 is a schematic diagram of the Stickland fermentation pathway in accordance with an example of the present invention.

FIG. 6 is a schematic diagram of a pMOL30 plasmid of Burkholderiales in accordance with another example of the present invention.

FIG. 7 is a schematic diagram showing a xanthan gum molecule binding to metal ions in accordance with yet another example of the present invention.

FIG. 8 is a heat map of OTUs from a sequencing report of an example rare earth metal extracting bacterial consortium in accordance with still another example of the present invention.

FIG. 9 is Venn diagram representing the number of OTUs present in four samples, at least one of which includes a rare earth metal extracting bacterial consortium in accordance with an example of the present invention.

FIGS. 10-15 are bar charts showing the relative abundance of the 30 most abundant classifications in each of four samples at different taxonomic levels from a sequencing report of an example rare earth metal extracting bacterial consortium in accordance with still another example of the present invention.

FIGS. 16-21 are heat maps showing the top 30 most abundant classifications in each sample at different taxonomic levels from a sequencing report of an example rare earth metal extracting bacterial consortium in accordance with still another example of the present invention.

FIGS. 22-23 are bar graphs showing estimated abundance of species in sample 1 and sample 4 in accordance with another example of the present invention.

FIG. 24 is a bar graph showing the portions of species that were unclassified, classified with confidence, and classified without confidence in sample 1 and sample 4 in accordance with another example of the present invention.

FIGS. 25-26 are bar graphs showing estimated abundance of genera in sample 1 and sample 4 in accordance with another example of the present invention.

FIG. 27 is a bar graph showing the portions of genera that were unclassified, classified with confidence, and classified without confidence in sample 1 and sample 4 in accordance with another example of the present invention.

Reference will now be made to the exemplary embodiments illustrated, and specific language will be used herein to describe the same. It will nevertheless be understood that no limitation of the scope of the invention is thereby intended.

### DETAILED DESCRIPTION

Before invention embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of compositions, dosage forms, treatments, etc., to provide a thorough understanding of various invention embodiments. One skilled in the relevant art will recognize, however, that such detailed embodiments do not limit the overall inventive concepts articulated herein, but are merely representative thereof.

### Definitions

It should be noted that as used herein, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. “Directly coupled” objects, structures, elements, or features are in contact with one another and may be attached. Further as used in this written description, it is to be understand that when using the term “coupled” support is also afforded for “directly coupled” and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Embodiments

An initial overview of technology embodiments is provided below and then specific technology embodiments are described in further detail later. This initial summary is intended to aid readers in understanding the technology more quickly but is not intended to identify key features or essential features of the technology nor is it intended to limit the scope of the claimed subject matter.

The present disclosure describes bacterial consortia that can be used to extract rare earth metals from a rare earth metal source (e.g. materials containing the rare earth metals). The bacterial consortia can be used to extract rare earth metals from a variety of rare earth metal sources, such as ore, minerals, industrial waste, electronic waste, and others. The rare earth metal source (“REMS”) can be any material that contains rare earth metal in a detectible concentration. In certain particular examples, the bacterial consortia can be used to extract rare earth metals from electronic waste. In more specific examples, the rare earth metal can include neodymium. In further examples, the bacterial consortia can be used to extract neodymium from magnet waste.

Current processes for recycling rare earth magnets (NdFeB alloy) are energy intensive and involve hazardous chemicals. For example, one process includes grinding the NdFeB alloy to a smaller particle size, heating the NdFeB alloy at a high temperature above 900° C., leaching with caustic agents such as hydrochloric acid and nitric acid, calcination, leaching with water, and ultrasonic spray pyrolysis. This process includes multiple energy intensive steps and hazardous chemicals.

The bacterial consortia described herein can be capable of or otherwise operable to extract rare earth metals from a REMS simply by incubating the bacteria in the presence of the REMS, such as in an aqueous medium with the REMS submerged or in contact with the medium. The ability of the bacterial consortia to survive, grow, and extract rare earth metals in such an environment is surprising. Many metals are toxic to most bacteria when the metals are present at a high concentration. Electronic waste in particular can include a large amount of various metals. Thus, using bacterial agents in recycling of electronic waste has often been difficult or impossible because the metals in the electronic waste kill the bacteria. Similarly, the direct use of live bacteria in recycling rare earth magnets would usually be impossible because the neodymium, iron, and other metals in the rare earth magnets would kill the bacteria. However, the bacterial consortia described herein have surprisingly been found to have a high resistance to these metals. The bacterial consortia have been shown to be capable of extracting neodymium from rare earth magnets by direct incubation of the bacteria in the presence of the rare earth magnets.

In various examples, the bacterial consortia can include a variety of different bacterial species. In some examples, a bacterial consortium can include at least the following types of bacteria: an acid secreting bacterium; a heavy metal resistant bacterium; an iron-sequestering molecule secreting bacteria; and a rare earth metal sequestering bacterium. In certain examples, each of the functions (acid secreting, heavy metal resisting, iron-sequestering molecule secreting, and rare earth metal sequestering) can be performed by a different single species of bacteria, or by. However, in other examples, the consortium can include multiple different bacterial species that perform one or more of the functions. For example, the consortium can include two, three, four, or more different bacterial species that secrete acids. Similarly, the consortium can include multiple bacterial species that are heavy metal resistant, or that secrete iron-sequestering molecules, or that sequester rare earth metals. The consortium can also include a bacterial species that performs more than one of these functions. For example, a single bacterial species can sequester rare earth metals and also secrete iron-sequestering molecules. Therefore, in some examples, any combination of the four types of bacteria listed above (the acid secreting bacterium, heavy metal resistant bacterium, iron-sequestering molecule secreting bacterium, and rare earth metal sequestering bacterium) may refer to a single bacterial species that performs a combination of these functions. Thus, the bacterial consortia described herein can include example four bacterial species, or less than four bacterial species, or more than four bacterial species, so long as the functions listed above are performed by bacteria in the consortium.

As used herein, “consortium” refers to a group, collection, or assembly of multiple bacteria types. In some cases, a consortium of bacteria can work together to perform a function that the individual bacteria species would not be capable of performing alone. The term “consortia” is used as the plural form of “consortium.” The various bacteria and features described herein can be included in a variety of combinations in a single bacterial consortium. Thus, multiple different bacterial consortia are described by the present disclosure. In the particular consortia described herein, the bacteria can work together to extract rare earth metals from a REMS. The bacteria in a consortium can be living in sufficiently close proximity that the bacteria can work together in some way. For example, the bacteria in the rare earth metal extracting consortia can be in sufficiently close proximity that if the consortium is incubated with a REMS, the acid secreting bacteria can secrete acid that leaches rare earth metal from the REMS, and the heavy metal resistant bacteria and iron-sequestering molecule secreting bacteria can provide protection for the consortium from the high concentration of metal from the REMS, and the rare earth metal sequestering bacteria can sequester the rare earth metal. In some examples, the bacteria in the consortium can all be present in a growth media within a single container, and the bacteria can mix freely in the container.

In one example, a rare earth metal extracting bacterial consortium can include an acid secreting bacterium, a heavy metal resistant bacterium, an iron-sequestering molecule secreting bacterium, and a rare earth metal sequestering bacterium. The acid secreting bacterium can be an organic acid secreting bacterium. In some examples, the organic acid secreting bacterium can be a citric acid secreting bacterium. The citric acid secreting bacterium can be Bacillus sp. In further examples, the organic acid secreting bacterium can be a butyric acid secreting bacterium. The butyric acid secreting bacterium can be Butyrivibrio hungatei. In other examples, the organic acid secreting bacteria can be an amino acid fermenting bacterium. The amino acid fermenting bacterium can be Clostridia venationis. Yet other bacterium/bacteria may be capable of secreting or otherwise producing other organic acids, such as ascorbic, lactic, gluconic, malonic, succinic, acetic, malic, oxalic, uric, or tartaric acids, etc.

The heavy metal resistant bacterium can resist heavy metal by active transport of metal ions, extracellular sequestration, intracellular sequestration, reduction of metal ions to insoluble metal, an extracellular barrier, or a combination thereof. In some examples, the heavy metal resistant bacterium can be from the order Burkholderiales or genus Cupriavidus. The heavy metal resistant bacterium can grow in the presence of a heavy metal concentration of 30 grams per liter or more. In certain examples, the heavy metal resistant bacterium can contain a plasmid with at least 99% sequence identity to the pMOL30 plasmid of Burkholderiales.

The iron-sequestering molecule secreting bacterium can secrete an iron-sequestering protein or an iron-sequestering siderophore, in some examples. In certain examples, the iron-sequestering molecule secreting bacterium can be capable of or otherwise operable to secrete at least 10 grams of iron-sequestering protein per 10¹² bacterial cells. In certain examples, the iron-sequestering molecule secreting bacterium can be Acinetobacter baumanni. In further examples, the rare earth metal sequestering bacterium can sequester rare earth metals by intracellular sequestration, extracellular sequestration, conversion to an insoluble metal, sequestration into a glycocalyx, sequestration by a specific binding protein, or a combination thereof. The rare earth metal sequestering bacterium can be capable of or otherwise operable to sequester at least 10 grams of rare earth metal per 10¹² bacterial cells. In some examples, the rare earth metal sequestering bacterium can be a xanthan gum secreting bacterium. The xanthan gum secreting bacterium can be Xanthomonas vesicatoria. In other examples, the rare earth metal sequestering bacterium can be Peptostreptococcus anaerobius or a lactobacillus having a rare earth metal sequestering S-layer.

The rare earth metal extracting bacterial consortium can also include Collinsella aerofaciens. In further examples, the bacterial consortium can also include axonopodis, brevis, anaerobius, frisia, coli, guillouiae, parainfluenzae, oryziterrae, Subtilis, and others. In some examples, the acid secreting bacterium, the heavy metal resistant bacterium, the iron-sequestering molecule secreting bacterium, and the rare earth metal sequestering bacterium can be aerobic bacteria. In other examples, the acid secreting bacterium, the heavy metal resistant bacterium, the iron-sequestering molecule secreting bacterium, and the rare earth metal sequestering bacterium can be anaerobic bacteria. A ratio of the number of acid secreting bacteria to heavy metal resistant bacteria to iron-sequestering molecule secreting bacteria to rare earth metal sequestering bacteria can be 1-100 acid secreting bacteria to 1-100 heavy metal resistant bacteria to 1-100 iron-sequestering molecule secreting bacteria to 1-100 rare earth metal sequestering bacteria.

In another example of the present disclosure, a composition can include a growth medium and a bacterial consortium growing in the growth medium. The growth media can include water, magnesium sulfate, manganese chloride, cobalt chloride, calcium chloride, ammonium sulfate, soluble starch, and amino acids. The bacterial consortium can include: an acid secreting bacterium, a heavy metal resistant bacterium, an iron-sequestering molecule secreting bacterium, and a rare earth metal sequestering bacterium. In various examples, the magnesium sulfate can be 0.01 wt % to 1 wt % of the growth medium; manganese chloride can be 0.01 wt % to 1 wt % of the growth medium; cobalt chloride can be 0.01 wt % to 1 wt % of the growth medium; calcium chloride can be 0.01 wt % to 1 wt % of the growth medium; ammonium sulfate can be 0.01 wt % to 1 wt % of the growth medium; soluble starch can be 0.01 wt % to 1 wt % of the growth medium; and amino acids can be 0.01 wt % to 1 wt % of the growth medium.

An example method of growing the bacterial consortium in the composition described above can include maintaining the composition at a temperature from about 25° C. to about 50° C. The method can also include aerating the composition. The method can also include feeding the bacterial consortium with new growth medium at a rate from about 0.5 mL per 100 mL of the composition per hour to about 2 mL per 100 mL of the composition per hour.

### Rare Earth Metal Extracting Bacterial Consortia

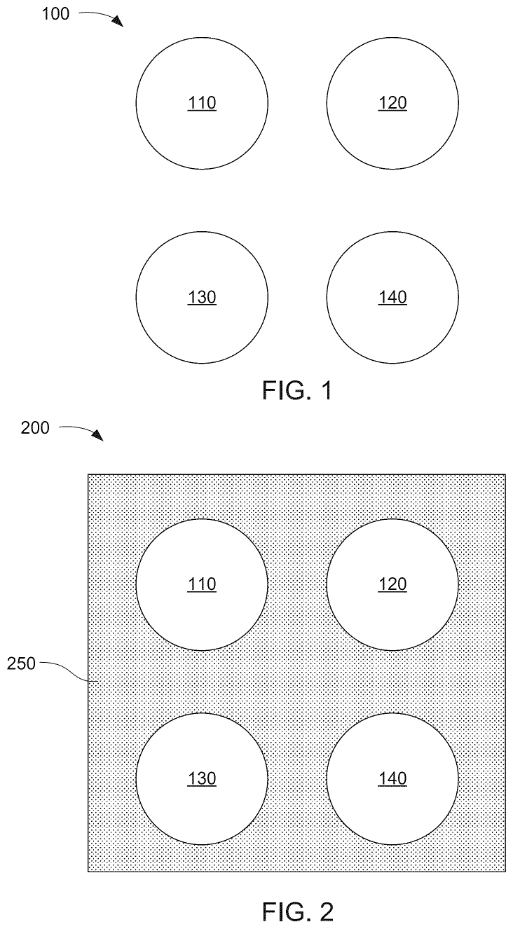

FIG. 1 is a schematic representation of one example of a rare earth metal extracting bacterial consortium 100 as described herein. The consortium includes an acid secreting bacterium 110, a heavy metal resistant bacterium 120, an iron-sequestering molecule secreting bacterium 130, and a rare earth metal sequestering bacterium 140. Specific examples of the various bacteria in the consortia are described in more detail below. Although the bacteria are described herein using the singular term “bacterium,” the consortia can include many bacterial cells of each type of bacterium. For example, “acid secreting bacterium” refers to a particular bacterial species that secretes acid, and many individual cells of this bacterial species can be present in the consortium. Additionally, as mentioned above, a consortia can include multiple different bacterial species that perform the same function, and/or a single bacterial species that performs multiple functions. The functions referred to include acid secretion, heavy metal resistance, iron-sequestering molecule secretion, and rare earth metal sequestration.

The rare earth metal extracting bacterial consortium can be capable of or otherwise operable to extract a rare earth metal from a REMS. In some examples, the bacteria of the consortium can be present in an amount sufficient to extract a rare earth metal from a material that includes the rare earth metal and another material. The amounts of bacteria can be expressed in various way, including the total number of bacterial cells in the consortium, the number ratio of each bacterial species in the consortium relative to other bacterial species in the consortium, the total concentration of bacterial cells (for example, if the consortium is in an aqueous medium), the concentration ratio of each bacterial species in the consortium relative to the concentrations of other bacterial species in the consortium, the amounts of each bacterial species that are sufficient for the individual species to perform their individual functions, and so on.

In certain examples, a rare earth metal extracting bacterial consortium can include multiple bacterial species in an effective amount or concentration to extract a rare earth metal from a REMS, wherein the multiple bacterial species include an acid secreting bacterium, a heavy metal resistant bacterium, an iron-sequestering molecule secreting bacterium, and a rare earth metal sequestering bacterium. The bacterial consortium can be in an aqueous medium and the concentration can be from 10² cells/mL to 10¹² cells/mL in some examples, or from 10³ cells/mL to 10¹¹ cells/mL, or from 10⁴ cells/mL to 10¹⁰ cells/mL, or from 10⁶ cells/mL to 10¹¹ cells/mL, or from 10⁸ cells/mL to 10¹¹ cells/mL in further examples. In some examples, these concentration values can refer specifically to the acid secreting bacterium, the heavy metal resistant bacterium, the iron-sequestering molecule secreting bacterium, and the rare earth metal sequestering bacterium, excluding any other types of bacteria that may be present. In other examples, these concentration values can include other types of bacteria present in the aqueous medium.

In other examples, a rare earth metal extracting bacterial consortium can include multiple bacterial species in effective number ratios or concentration ratios to extract a rare earth metal from a REMS, wherein the multiple bacterial species include an acid secreting bacterium, a heavy metal resistant bacterium, an iron-sequestering molecule secreting bacterium, and a rare earth metal sequestering bacterium. A ratio of these bacterial species can be expressed as “A:B:C:D” where A is the number or concentration of acid secreting bacteria, B is the number or concentration of heavy metal resistant bacteria, C is the number or concentration of iron-sequestering molecule secreting bacteria, and D is the number or concentration of rare earth metal sequestering bacteria. Each relative number or concentration of bacteria can vary with a range, such as from 1 to 100. In certain examples, the ratio A:B:C:D can be 1-100:1-100:1-100:1-100. In other words, the amount of each type of bacteria can be up to 100 times the amount of any other type of bacteria in the ratio. In further examples, the ratio can be 1-50:1-50:1-50:1-50, or 1-20:1-20:1-20:1-20, or 1-10:1-10:1-10:1-10, or 1-5:1-5:1-5:1-5. In further examples, the acid secreting bacteria can be more abundant than the other bacterial species in the consortium. In such examples, the ratio can be 100-200:1-100:1-100:1-100 or 50-100:1-50:1-50:1-50 or 10-20:1-10:1-10:1-10 or 5-10:1-5:1-5:1-5.

In further examples, a rare earth extracting bacterial consortium can include: an acid secreting bacterium in an effective amount to secrete sufficient acid to leach a rare earth metal from a REMS; a heavy metal resistant bacterium in an effective amount to provide resistance to the consortium to toxicity from the leached rare earth metal and other metals leached from the REMS; an iron-sequestering molecule secreting bacterium in an effective amount to secrete sufficient iron-sequestering molecule to protect the consortium from iron leached from the REMS; and a rare earth metal sequestering bacterium in an effective amount to sequester at least some of the rare earth metal leached from the REMS.

The rare earth metal extracting bacterial consortium can also be capable of or otherwise operable to extract a rare earth metal at a particular rate. In certain examples, a rare earth metal extracting bacterial consortium can include multiple bacterial species in an aqueous medium in effective amounts or concentrations to extract rare earth metal from a REMS at a rate from 0.1 grams of rare earth metal per day per liter to 10 grams per day per liter. The multiple bacterial species can include an acid secreting bacterium, a heavy metal resistant bacterium, an iron-sequestering molecule secreting bacterium, and a rare earth metal sequestering bacterium. In further examples, the bacteria can be present in effective amounts or concentrations to extract the rare earth metal at a rate from 0.3 grams per day per liter to 7 grams per day per liter, or from 0.5 grams per day per liter to 5 grams per day per liter, or from 0.5 grams per day per liter to 10 grams per day per liter.

The bacterial consortia described herein can be capable of or otherwise operable to extract rare earth metals from a variety of rare earth metal sources (REMS). Some such sources can include a rare earth metal and one or more additional metals. In certain examples, the REMS can include a rare earth metal and iron. The iron-sequestering molecule secreting bacteria can be particularly useful for sequestering iron to protect the consortium from toxicity due to iron present in the material. Rare earth magnets are one particular example of a material that includes a rare earth metal (neodymium) and iron. The bacterial consortia can be capable of or otherwise operable to extract neodymium from rare earth magnets.

Rare earth metals that can be extracted using the bacterial consortia include scandium, yttrium, lanthanum, cerium, praseodymium, neodymium, promethium, samarium, europium, gadolinium, terbium, dysprosium, holmium, erbium, thulium, ytterbium, and lutetium. Rare earth metals can be present in a variety of materials that include other metals and nonmetals. The amount of rare earth metal in these materials can vary depending on the material, from a few parts per million to 50 wt % or more. Rare earth magnets can include neodymium in an amount from about 29 wt % to about 32 wt %. Many other elements can be present in the material in addition to the rare earth metal. For example, rare earth magnets can include iron in an amount from about 64.2 wt % to about 68.5 wt %, boron in an amount from about 1 wt % to 1.2 wt %, and aluminum in an amount from about 0.2 wt % to about 0.4 wt %. Besides rare earth magnets, other materials from which rare earth metals can be extracted can include ore, minerals, industrial waste, electronic waste, magnet waste, printed circuit boards, lasers, electronic displays, batteries, computer memory, processors, light bulbs, light emitting diodes, and others. In various examples, the material from which rare earth metals are extracted can have a rare earth metal content from 100 ppm by weight to 75 wt %, or from 1000 ppm by weight to 50 wt %, or from 1 wt % to 50 wt %, or from 1 wt % to 20 wt %, or from 1 wt % to 10 wt %, or from 20 wt % to 50 wt %, or from 30 wt % to 50 wt %.

As mentioned above, the bacterial consortia can be in an aqueous medium. In certain examples, the aqueous medium can include a growth medium including nutrients that can be used by the bacteria in the consortium to allow the bacteria to multiply. FIG. 2 shows a schematic representation of a composition 200 that includes a growth medium 250 with bacteria in the growth medium. The bacteria include an acid secreting bacterium 110, a heavy metal resistant bacterium 120, an iron-sequestering molecule secreting bacterium 130, and a rare earth metal sequestering bacterium.

Non-limiting examples of ingredients that can be in the growth medium include water, sodium phosphate, sodium molybdate, sodium chloride, manganese chloride, ammonium chloride, cobalt chloride, tripotassium citrate, trisodium citrate, dipotassium hydrogen phosphate, potassium dihydrogen phosphate, calcium chloride, magnesium sulfate, ammonium sulfate, sodium acetate, dextrose, glucose, soluble starch, and amino acids. In various examples, the growth medium can include tripotassium citrate in an amount from about 0.01 wt % to about 1 wt %, or from about 0.05 wt % to about 1 wt %, or from about 0.1 wt % to about 1 wt %, or from about 0.05 wt % to about 0.5 wt %, or from about 0.01 wt % to about 0.5 wt %. Trisodium citrate can be included in an amount from about 0.01 wt % to about 1 wt %, or from about 0.1 wt % to about 1 wt %, or from about 0.1 wt % to about 0.5 wt %, or from about 0.05 wt % to about 0.5 wt %. Dipotassium hydrogen phosphate can be included in an amount from about 0.01 wt % to about 1.5 wt %, or from about 0.5 wt % to about 1.5 wt %, or from about 0.5 wt % to about 1.0 wt %, or from about 0.1 wt % to about 1.0 wt %. Potassium dihydrogen phosphate can be included in an amount from about 0.01 wt % to about 1 wt %, or from about 0.1 wt % to about 1 wt %, or from about 0.1 wt % to about 0.5 wt %, or from about 0.05 wt % to about 0.5 wt %. Manganese chloride can be included in an amount from about 0.01 wt % to about 1 wt %, or from about 0.1 wt % to about 1 wt %, or from about 0.1 wt % to about 0.5 wt %, or from about 0.05 wt % to about 0.5 wt %. Cobalt chloride can be included in an amount from about 0.01 wt % to about 1 wt %, or from about 0.1 wt % to about 1 wt %, or from about 0.1 wt % to about 0.5 wt %, or from about 0.05 wt % to about 0.5 wt %. Calcium chloride can be included in an amount from about 0.01 wt % to about 1 wt %, or from about 0.05 wt % to about 1 wt %, or from about 0.05 wt % to about 0.5 wt %, or from about 0.05 wt % to about 0.2 wt %, or from about 0.01 wt % to about 0.2 wt %. Magnesium sulfate can be included in an amount from about 0.01 wt % to about 5 wt %, or from about 0.01 wt % to about 3 wt %, or from about 0.1 wt % to about 3 wt %, or from about 0.1 wt % to about 5 wt %. Ammonium sulfate can be included in an amount from about 0.01 wt % to about 1 wt %, or from about 0.01 wt % to about 0.5 wt %, or from about 0.01 wt % to about 0.2 wt %, or from about 0.05 wt % to about 1 wt %, or from about 0.05 wt % to about 0.5 wt %. Sodium acetate can be included in an amount from about 0.01 wt % to about 5 wt %, or from about 0.01 wt % to about 3 wt %, or from about 0.1 wt % to about 3 wt %, or from about 0.1 wt % to about 5 wt %. Soluble starch can be included in an amount from about 0.01 wt % to about 1 wt %, or from about 0.1 wt % to about 1 wt %, or from about 0.1 wt % to about 0.5 wt %, or from about 0.05 wt % to about 0.5 wt %. Dextrose can be included in an amount from about 0.01 wt % to about 1 wt %, or from about 0.01 wt % to about 0.5 wt %, or from about 0.01 wt % to about 0.2 wt %, or from about 0.05 wt % to about 1 wt %, or from about 0.05 wt % to about 0.5 wt %. Glucose can be included in an amount from about. 01 wt % to about 5 wt %, or from about 0.01 wt % to about 1 wt %, or from about 0.01 wt % to about 0.2 wt %, or from about 0.05 wt % to about 1 wt %, or from about 0.05 wt % to about 0.5 wt %. Amino acids can be included in an amount from about 0.01 wt % to about 1 wt %, or from about 0.1 wt % to about 0.6 wt %, or from about 0.3 wt % to about 0.6 wt %, or from about 0.3 wt % to about 1 wt %.

The bacterial consortium can be grown in the medium described above by maintaining the bacteria at a suitable temperature for growth. In some examples, the bacteria can be incubated in the medium at a temperature from about 20° C. to about 90° C., or from about 25° C. to about 50° C. If the consortium includes aerobic bacteria, then the composition can be aerated to provide oxygen to the aerobic bacteria. The bacterial consortium can also be fed by adding additional growth medium at a sufficient rate to provide nutrients for the growing bacteria. In some examples, additional growth medium can be added at a rate from about 0.5 mL per 100 mL of the composition per hour to about 2 mL per 100 mL of composition per hour.

Other bacterial species may also be useful to include in the consortium. Additional bacterial species can perform various functions in the consortium. In some examples, the consortium can also include Collinsella aerofaciens, which can be useful for converting glucose to butyrate. Additional bacterial species that can be in the consortium include axonopodis, brevis, anaerobius, frisia, coli, guillouiae, parainfluenzae, oryziterrae, Subtilis, and others. The bacteria in the consortia are described in more detail below.

The bacterial consortium can extract rare earth metal from a REMS most effectively when the bacterial consortium is in a stationary phase. The stationary phase can be a stage when growth of the bacteria is reduced or stopped, but the bacterial cells remain metabolically active. In other words, the concentration of bacterial cells stops increasing or increases at a reduced rate while the cells remain metabolically active. The occurrence of stationary phase can depend on a variety of factors, including the availability of nutrients for the bacterial cells that are present. In some examples, the bacterial consortia described herein can reach stationary phase when the concentration of bacteria has reached a threshold from about 10⁹ cells per mL to about 10¹¹ cells per mL, or in some examples around 10¹⁰ cells per mL. Accordingly, in some examples the bacteria can multiply, increasing the concentration of bacterial cells, until the concentration reaches this threshold at which point the growth of the number of cells can slow or stop, but the cells can remain metabolically active. The metabolic activity of the cells can include secreting the compounds and performing the functions described above.

### Acid Secreting Bacteria

The rare earth metal extracting bacterial consortia described herein can include an acid secreting bacterium. The acid secreting bacteria can secret an acid that is capable of or otherwise operable to leach rare earth metal from a REMS. In some examples, the acid secreting bacterium can be a single bacterial species that secretes a single acidic compound. In other examples, the acid secreting bacterium can be a single bacterial species that secretes multiple acidic compounds. In further examples, the acid secreting bacterium can include multiple bacterial species that secrete one or more acidic compounds. Some acid secreting bacterial species can be aerobic bacteria, while others can be anaerobic bacteria. The consortia described herein can include either aerobic or anaerobic acid secreting bacteria. In some examples, the consortium can include both an aerobic acid secreting bacterium and an anaerobic acid secreting bacterium. This can be useful to allow the consortium to be used is both aerobic and anaerobic conditions.

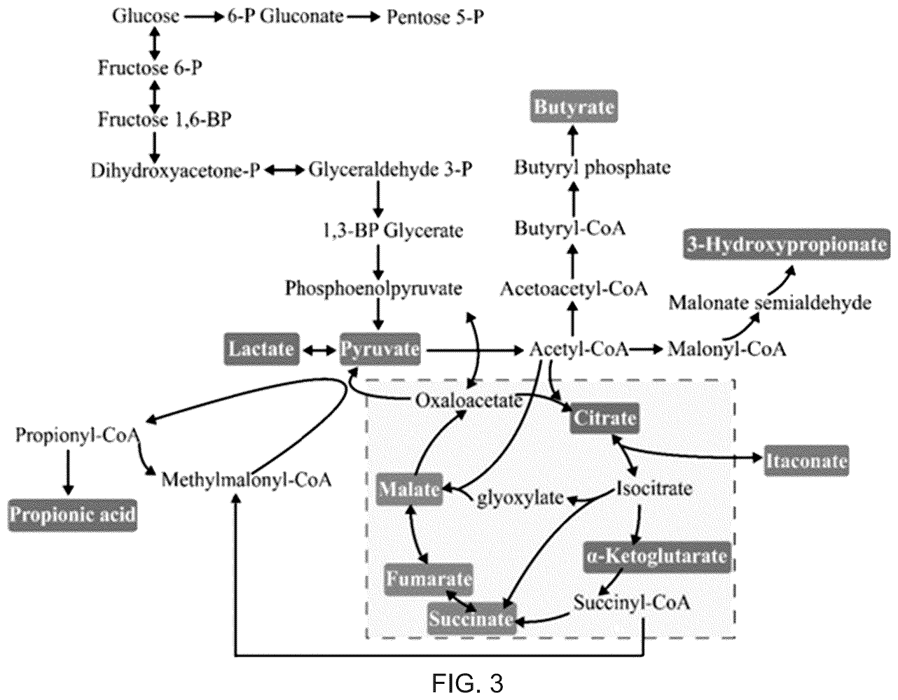

In some examples, the acid secreting bacterium can be an organic acid secreting bacterium. One type of organic acid secreting bacterium that can be used is a citric acid secreting bacterium, such as Bacillus sp. FIG. 3 is a schematic diagram of a metabolic pathway used by Bacillus sp. to produce citric acid.

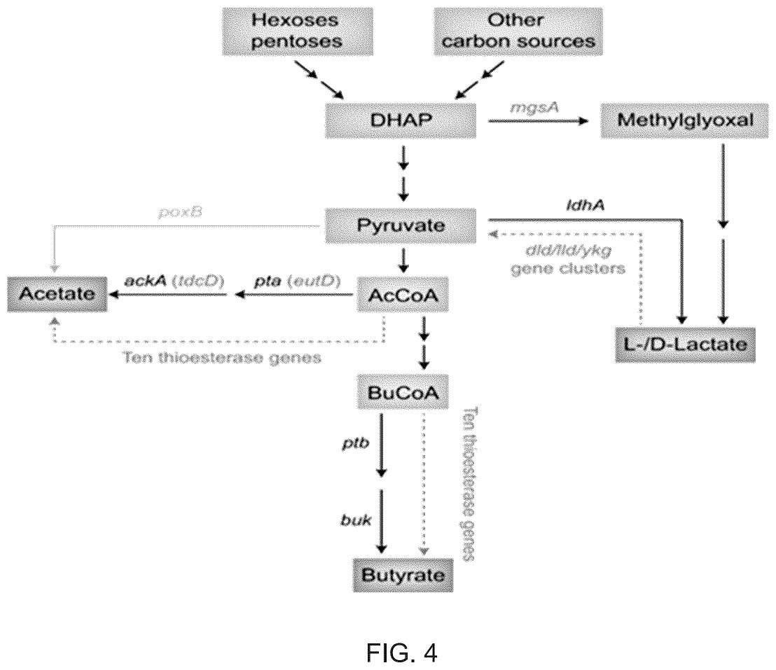

The organic acid secreting bacterium can also include a butyric acid secreting bacterium. One example of such a bacterium is Butyrivibrio hungatei. FIG. 4 is a schematic diagram of a metabolic pathway used by Butyrivibrio hungatei to produce butyric acid.

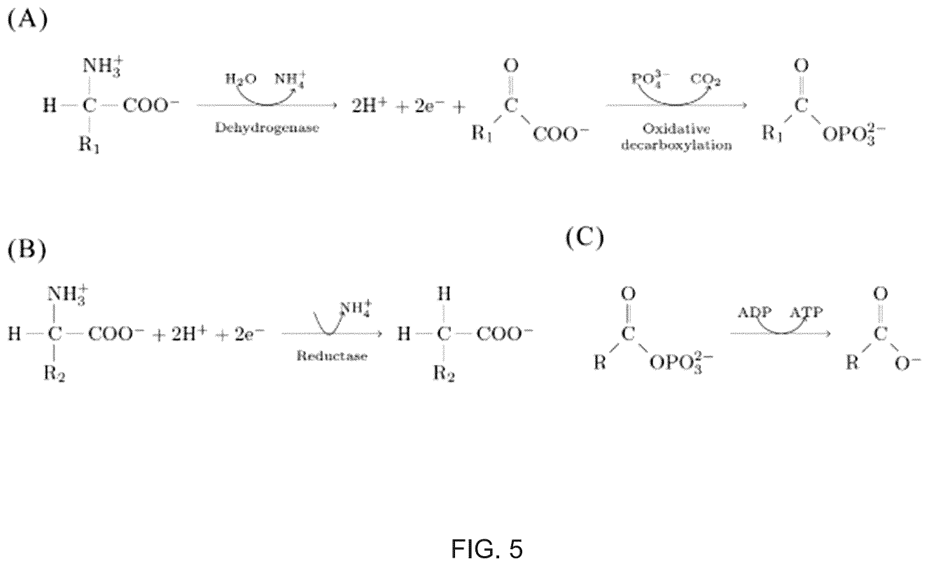

In still further examples, the organic acid secreting bacterium can include an amino acid fermenting bacterium. One pathway for amino acid fermentation is referred to as “Stickland fermentation.” An example of a bacterium that utilizes this pathway is Clostridia venationis. FIG. 5 is a schematic diagram of the Stickland fermentation pathway. In this pathway, an amino acid is converted to organic acids.

The acid secreting bacteria can be capable of or otherwise operable to secrete a sufficient amount of acid to leach the rare earth metal from a REMS. In some examples, the acid secreting bacteria can secret acid in an amount from about 0.1 gram per liter of the consortium in an aqueous medium per day to about 100 grams per liter per day, or from about 0.1 grams per liter per day to about 30 grams per liter per day, or from about 1 grams per liter per day to about 10 grams per liter per day. The acid produced by the acid secreting bacteria can reduce the pH of the aqueous medium in which the bacterial consortium grows. In some examples, the pH of the composition including the aqueous medium and the bacterial consortium can be from about 3 to about 6, or from about 3.5 to about 5.5, or from about 3.5 to about 4.5, or from about 4.5 to about 6, or from about 4.5 to about 5.5.

### Heavy Metal Resistant Bacteria

The rare earth metal extracting bacterial consortium can also include a heavy metal resistant bacterium. The heavy metal resistant bacterium can provide protection for the consortium from toxicity of high concentrations of metals. In some examples, the heavy metal resistant bacterium can be a single bacteria that provides heavy metal resistance. In other examples, the heavy metal resistant bacterium can include multiple bacterial species that provide heavy metal resistance. The heavy metal resistant bacterium can also be an aerobic bacterium or an anaerobic bacterium. In certain examples, the heavy metal resistant bacterium can include both an aerobic bacterium and an anaerobic bacterium so that the consortium can have heavy metal resistance in either aerobic conditions or anaerobic conditions.

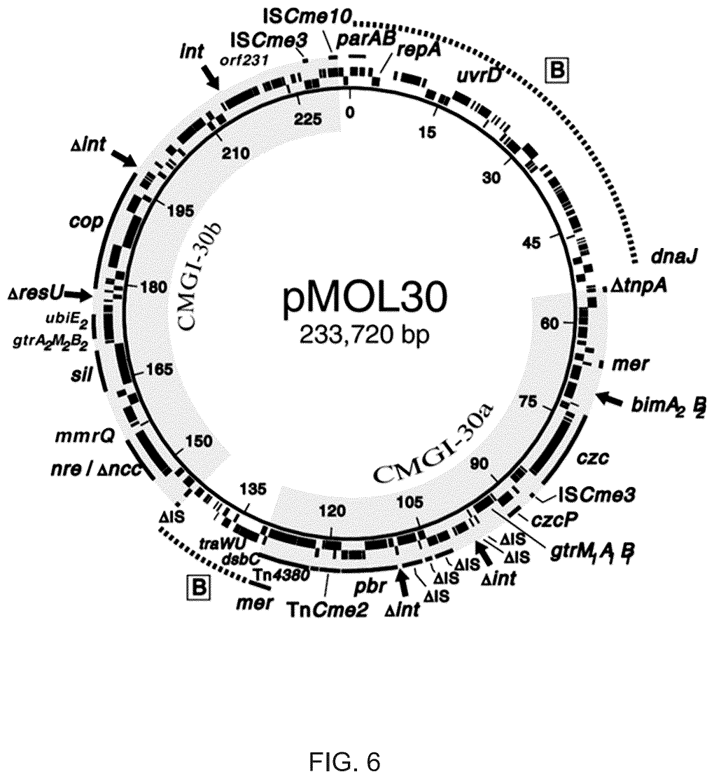

Heavy metal resistant bacteria can utilize several mechanisms for metal resistance, including active transport of metal ions, extracellular sequestration, intracellular sequestration, reduction of metal ions to insoluble metal, and extracellular barriers. Genetic determinants of heavy metal resistance can be localized both on bacterial chromosomes and on extrachromosomal genetic elements. Horizontal gene transfer can allow the heavy metal resistant bacteria to spread the characteristic of heavy metal resistance to other bacteria. In some cases heavy metal resistance can be provided by a plasmid contained by the bacteria. Examples of heavy metal resistant bacteria include bacteria of the order Burkholderiales and bacteria of the genus Cupriavidus. Bacteria of the order Burkholderiales can include a pMOL30 plasmid, which is diagrammed in FIG. 6. In certain examples, the heavy metal resistant bacteria can include a plasmid having at least 99% sequence identity to the pMOL30 plasmid of Burkholderiales.

The heavy metal resistant bacteria can allow the bacterial consortium to grow in an aqueous medium that has a high concentration of metals. In some examples, the heavy metal resistant bacteria can be capable of or otherwise operable to grow in the presence of a heavy metal concentration of 30 grams per liter or more. In further examples, the heavy metal resistant bacteria can be capable of or otherwise operable to grow in the presence of a heavy metal concentration of up to 50 grams per liter or up to 40 grams per liter.

### Iron-Sequestering Molecule Secreting Bacteria

The rare earth metal extracting bacterial consortium can also include an iron-sequestering molecule or agent secreting bacterium. This bacterium can also help protect the consortium from high concentrations of iron by sequestering the iron ions. In some examples, a single iron-sequestering molecule secreting bacterium can produce a single type of iron-sequestering molecule. In other examples, multiple bacterial species can produce a single type of iron-sequestering molecule. In still further examples, multiple bacterial species can produce multiple different iron-sequestering molecules. Any of these combinations can be encompassed by the iron-sequestering molecule secreting bacterium. Examples of iron-sequestering molecules that can be secreted include iron-sequestering proteins and iron-sequestering siderophores. Examples of iron-sequestering siderophores include enterobactin, pyochelin, alcaligin, rhizoferrin, and others. The iron-sequestering molecule secreting bacterium can also be an aerobic bacterium or an anaerobic bacterium. In certain examples, the iron-sequestering molecule secreting bacterium can include both an aerobic bacterium and an anaerobic bacterium so that the consortium can sequester iron in either aerobic conditions or anaerobic conditions.

The iron-sequestering molecule secreting bacterium can produce at least 10 grams of iron-sequestering protein per 10¹² bacterial cells in some examples. In further examples, the iron-sequestering molecule secreting bacterium can produce at least 20 grams, or at least 30 grams, or at least 40 grams of iron-sequestering protein per 10¹² bacterial cells. An example iron-sequestering molecule secreting bacterium is Acinetobacter Baumannii.

### Rare Earth Metal Sequestering Bacteria

The rare earth metal extracting bacterial consortium can also include a rare earth metal sequestering bacterium. This bacterium can sequester rare earth metal that has been liberated from a material, which can both help protect the bacterial consortium from toxicity of the rare earth metal and help prepare the rare earth metal to be recovered, separated and purified for further use. In some examples, the bacterial consortium can include a single rare earth metal sequestering bacterial species. In other examples, the bacterial consortium can include multiple different rare earth metal sequestering bacterial species. The rare earth metal sequestering bacteria can sequester rare earth metals selectively or non-selectively. The rare earth metal sequestering bacterium can also be an aerobic bacterium or an anaerobic bacterium. In certain examples, the rare earth sequestering bacterium can include both an aerobic bacterium and an anaerobic bacterium so that the consortium can sequester rare earth metals in either aerobic conditions or anaerobic conditions.

Rare earth metal sequestering bacteria can sequester rare earth metals using several mechanisms. Potential metal sequestration mechanisms include intracellular sequestration, extracellular sequestration, conversion into an insoluble metal, sequestration into a glycocalyx, and sequestration with specific binding proteins. Some bacteria can sequester metal by surface adsorption. These bacteria can include surface groups on their cell walls that can adsorb rare earth metals. Carboxyl groups and phosphoryl groups are examples of surface functional groups that can bind to metal nonspecifically. In certain examples, cells can include selective binding groups such as selective peptides and proteins on the cell wall. These can selectively bind to certain metals. The selective surface groups can be naturally occurring, or they may be enabled by genetic engineering of the bacteria to produce such surface groups on the outside of the cell wall. The bacteria can include surface groups that selectively bind neodymium in some examples. If other specific rare earth metals are desired then the bacteria can include surface groups the specifically bind to those specific rare earth metals.

The rare earth metal sequestering bacteria can be capable of or otherwise operable to sequester rare earth metal at a rate of at least 10 grams of rare earth metal per 10¹² bacterial cells. In further examples, the rare earth metal sequestering bacteria can be capable of or otherwise operable to sequester rare earth metal at a rate of at least 15 grams or at least 20 grams of rare earth metal per 10¹² bacterial cells. In certain examples, the rare earth metal sequestering bacteria can sequester the rare earth metals using a glycocalyx or S-layer on the cell wall. Examples of bacteria that can sequester rare earth metals in this way include Peptostreptococcus anaerobius and lactobacillus having a rare earth metal sequestering S-layer. These can sequester rare metals specifically or nonspecifically as explained above. In certain examples, the bacteria can be capable of or otherwise operable to selectively sequester one or more rare earth metals selected from the group consisting of scandium, yttrium, lanthanum, cerium, praseodymium, neodymium, promethium, samarium, europium, gadolinium, terbium, dysprosium, holmium, erbium, thulium, ytterbium, and lutetium.

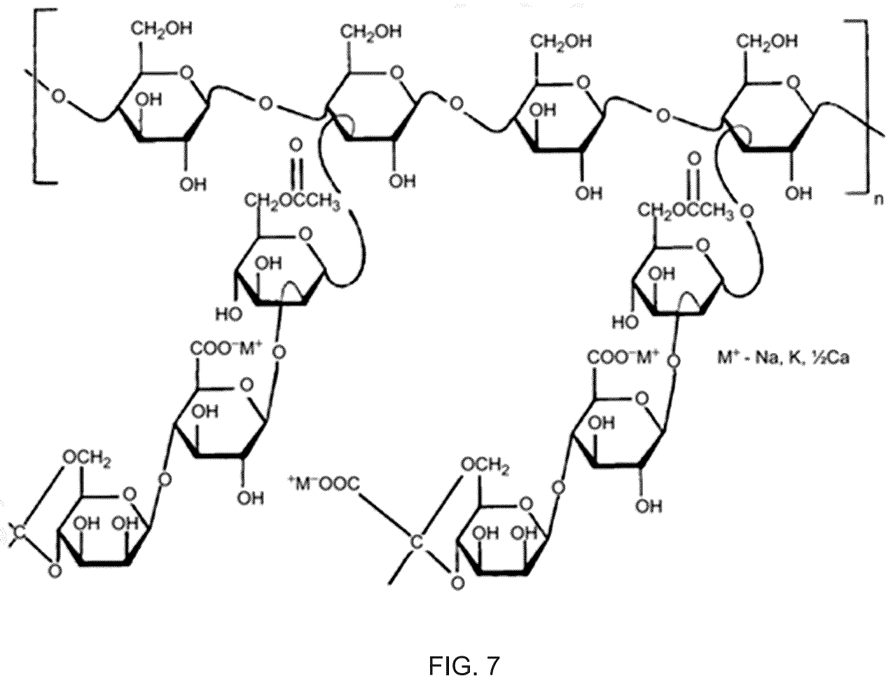

Some rare earth metal sequestering bacteria can secrete a molecule that binds to the rare earth metal, either specifically or nonspecifically. Xanthan gum is one example of a molecule that can bind to rare earth metals nonspecifically. In some examples, the rare earth metal sequestering bacterium can be a bacterium that secretes xanthan gum. FIG. 7 is a schematic diagram showing a xanthan gum molecule binding to metal ions. An example bacterial species that secretes xanthan gum is Xanthomonas vesicatoria. Other Xanthomonas species can also secrete xanthan gum. Additionally, rare earth metals can be bound by other negatively charged molecules that can be secreted by other bacteria.

It is to be understood that the examples of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular examples only and is not intended to be limiting.

Reference throughout this specification to “one example” or “an example” means that a particular feature, structure, or characteristic described in connection with the example is included in at least one example of the present invention. Thus, appearances of the phrases “in one example” or “in an example” in various places throughout this specification are not necessarily all referring to the same example.

Although the disclosure may not expressly disclose that some examples or features described herein may be combined or interchanged with other examples or features described herein, this disclosure should be read to describe any such combinations that would be practicable by one of ordinary skill in the art no matter the specific examples that were described. Indeed, unless a certain combination of elements or functions not expressly disclosed would conflict with one another, such that the combination would render the resulting example inoperable or impracticable as would be apparent to those skilled in the art, this disclosure is meant to contemplate that any disclosed element or feature or function in any example described herein can be incorporated into any other example described herein (e.g., the elements or features or functions combined or interchanged with other elements or features or functions across examples) even though such combinations or interchange of elements or features or functions and resulting examples may not have been specifically or expressly disclosed and described. The use of “or” in this disclosure should be understood to mean non-exclusive or, i.e., “and/or,” unless otherwise indicated herein.

As used herein, a plurality of items, structural elements, compositional elements, and/or materials can be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various examples of the present invention can be referred to herein along with alternatives for the various components thereof. It is understood that such examples and alternatives are not to be construed as de facto equivalents of one another, but are to be considered as separate and autonomous representations of the present invention.

Furthermore, the described features, structures, or characteristics can be combined in any suitable manner in one or more examples. In the description, numerous specific details are provided, such as examples of lengths, widths, shapes, etc., to provide a thorough understanding of examples of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

Concentrations, dimensions, amounts, and other numerical data may be presented herein in a range format. It is to be understood that such range format is used merely for convenience and brevity and should be interpreted flexibly to include the numerical values explicitly recited as the limits of the range, as well as to include all the individual numerical values or sub-ranges encompassed within that range as the individual numerical value and/or sub-range is explicitly recited. For example, a weight ratio range of about 1 wt % to about 20 wt % should be interpreted to include the explicitly recited limits of 1 wt % and 20 wt % and to include individual weights such as about 2 wt %, about 11 wt %, about 14 wt %, and sub-ranges such as about 10 wt % to about 20 wt %, about 5 wt % to about 15 wt %, etc.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

### EXAMPLES

A microbial consortium was prepared from a sample collected from a waste drain in a brewery. The microbes from the sample were incubated and grown in a growth medium including the ingredients shown in Table 1:

TABLE 1

| Ingredient | Weight Percent (wt %) |
|---|---|
| Tripotassium citrate (K₃H₅C₆O₇) | 0.05 |
| K₂HPO₄ | 0.7 |
| KH₂PO₄ | 0.2 |
| CaCl₂ | 0.07 |
| MgSO₄ | 2.5 |
| (NH₄)₂SO₄ | 0.1 |
| C₂H₃NaO₂ | 2.5 |
| Dextrose | 0.1 |
| Casamino acids | 0.5 |
| Water | balance |

The microbes from the sample were incubated in the growth medium in the presence of metals including copper and neodymium. The concentrations of metal in the growth medium were gradually increased over time to see the effect this would have on the bacteria in the sample.

Four separate sample portions were derived from the original sample. These are referred to as “sample 1,” sample 2,” “sample 3,” and “sample 4.” Samples 1 and 2 were both taken from the original sample, which was collected from the brewery waste drain. Sample 3 was taken part way through the treatment of the microbes by incubation in the presence of metals described above. Sample 4 was taken after the treatment by incubation in the presence of metals described above.

Samples 1-4 were analyzed using the GENEWIZ® 16S-EZ bioinformatics analysis process from GENEWIZ (USA). This process included PCR amplification of the V3 and V4 hypervariable regions of the 16S rDNA. The V3 and V4 hypervariable regions were then sequenced. The sequences were clustered into operational taxonomic units (OTUs) where each OTU was defined by a 97% sequence identity threshold. The taxonomy of each OTU was identified, if known. The abundance of each OTU in samples 1, 2, 3, and 4 and their taxonomy are listed in Table 2.

TABLE 2

| OTU | 1 | 2 | 3 | 4 | taxonomy |
|---|---|---|---|---|---|
| OTU1 | 940 | 532 | 336 | 0 | k_Bacteria |
| OTU2 | 848 | 436 | 355 | 0 | k_Bacteria |
| OTU3 | 110,010 | 114,917 | 91,114 | 101,000 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU4 | 270 | 143 | 82 | 0 | k_Bacteria |
| OTU5 | 140 | 151 | 164 | 92 | k_Bacteria |
| OTU6 | 227 | 130 | 64 | 0 | k_Bacteria |
| OTU7 | 115 | 131 | 129 | 88 | k_Bacteria |
| OTU8 | 68 | 65 | 97 | 34 | k_Bacteria |
| OTU9 | 277 | 142 | 82 | 0 | k_Bacteria |
| OTU10 | 100 | 125 | 131 | 0 | k_Bacteria |
| OTU11 | 119 | 61 | 36 | 0 | Unclassified |
| OTU12 | 136 | 45 | 24 | 0 | Unclassified |
| OTU13 | 52 | 54 | 61 | 46 | k_Bacteria |
| OTU14 | 0 | 0 | 0 | 271 | Unclassified |
| OTU15 | 239 | 138 | 75 | 0 | k_Bacteria |
| OTU16 | 49 | 57 | 43 | 47 | Unclassified |
| OTU17 | 0 | 0 | 0 | 208 | Unclassified |
| OTU18 | 52 | 50 | 48 | 48 | Unclassified |
| OTU19 | 190 | 98 | 38 | 0 | k_Bacteria |
| OTU20 | 172 | 89 | 47 | 0 | k_Bacteria |
| OTU21 | 32 | 41 | 14 | 17 | k_Bacteria |
| OTU22 | 2 | 0 | 51 | 46 | k_Bacteria |
| OTU23 | 0 | 38 | 36 | 0 | k_Bacteria |
| OTU24 | 0 | 48 | 0 | 30 | k_Bacteria |
| OTU25 | 0 | 0 | 0 | 81 | k_Bacteria; |
| | | | | | p_Proteobacteria; |
| | | | | | c_Alphaproteobacteria; |
| | | | | | o_Rickettsiales; |
| | | | | | f_mitochondria |
| OTU26 | 53 | 24 | 9 | 0 | Unclassified |
| OTU27 | 0 | 0 | 0 | 212 | k_Bacteria; |
| | | | | | p_Proteobacteria; |
| | | | | | c_Alphaproteobacteria; |
| | | | | | o_Rickettsiales; |
| | | | | | f_mitochondria |
| OTU28 | 0 | 39 | 0 | 32 | k_Bacteria |
| OTU29 | 52 | 25 | 11 | 0 | Unclassified |
| OTU30 | 56 | 18 | 11 | 0 | Unclassified |
| OTU31 | 119 | 102 | 91 | 80 | Unclassified |
| OTU32 | 0 | 110 | 57 | 0 | k_Bacteria |
| OTU33 | 1 | 37 | 19 | 40 | Unclassified |
| OTU34 | 186 | 106 | 36 | 88 | k_Bacteria; |
| | | | | | p_Bacteroidetes; |
| | | | | | c_Flavobacteriia; |
| | | | | | o_Flavobacteriales; |
| | | | | | f_Cryomorphaceae; |
| | | | | | g_; |
| | | | | | s_ |
| OTU35 | 37 | 19 | 10 | 0 | Unclassified |
| OTU36 | 1 | 1 | 26 | 56 | Unclassified |
| OTU37 | 0 | 0 | 0 | 277 | k_Bacteria |
| OTU38 | 0 | 28 | 36 | 0 | k_Bacteria |
| OTU39 | 0 | 2 | 0 | 206 | k_Bacteria |
| OTU40 | 0 | 0 | 0 | 29 | Unclassified |
| OTU41 | 0 | 8 | 6 | 12 | Unclassified |
| OTU42 | 0 | 31 | 0 | 0 | k_Bacteria |
| OTU43 | 0 | 34 | 16 | 25 | Unclassified |
| OTU44 | 3 | 2 | 29 | 34 | Unclassified |
| OTU45 | 225 | 226 | 8,160 | 8,808 | k_Bacteria; |
| | | | | | p_Firmicutes; |
| | | | | | c_Clostridia; |
| | | | | | o_Clostridiales; |
| | | | | | f_Lachnospiraceae; |
| | | | | | g_; |
| | | | | | s_ |
| OTU46 | 477 | 256 | 103 | 213 | k_Bacteria; |
| | | | | | p_Cyanobacteria; |
| | | | | | c_Synechococcophycideae; |
| | | | | | o_Synechococcales; |
| | | | | | f_Synechococcaceae |
| OTU47 | 47 | 0 | 0 | 0 | k_Bacteria |
| OTU48 | 186 | 108 | 28 | 93 | k_Bacteria; |
| | | | | | p_Proteobacteria; |
| | | | | | c_Alphaproteobacteria; |
| | | | | | o_Rhodobacterales; |
| | | | | | f_Rhodobacteraceae; |
| | | | | | g_Loktanella; |
| | | | | | s_ |
| OTU49 | 18 | 31 | 14 | 17 | k_Bacteria |
| OTU50 | 16 | 32 | 15 | 17 | k_Bacteria |
| OTU51 | 7 | 6 | 4,927 | 5,052 | k_Bacteria; |
| | | | | | p_Firmicutes; |
| | | | | | c_Clostridia; |
| | | | | | o_Clostridiales; |
| | | | | | f_Peptostreptococcaceae; |
| | | | | | g_Clostridium; |
| | | | | | s_venationis |
| OTU52 | 5 | 6 | 10 | 0 | k_Bacteria |
| OTU53 | 21 | 6 | 7 | 0 | Unclassified |
| OTU54 | 0 | 17 | 0 | 15 | k_Bacteria |
| OTU55 | 35 | 0 | 0 | 0 | k_Bacteria |
| OTU56 | 33 | 0 | 0 | 0 | k_Bacteria |
| OTU57 | 11 | 7 | 4 | 0 | k_Bacteria |
| OTU58 | 12 | 5 | 4 | 0 | k_Bacteria |
| OTU59 | 0 | 0 | 21 | 0 | Unclassified |
| OTU60 | 8 | 0 | 0 | 37 | Unclassified |
| OTU61 | 91 | 130 | 121 | 581 | k_Bacteria; |
| | | | | | p_Proteobacteria; |
| | | | | | c_Gammaproteobacteria; |
| | | | | | o_Xanthomonadales; |
| | | | | | f_Xanthomonadaceae; |
| | | | | | g_Xanthomonas; |
| | | | | | s_axonopodis |
| OTU62 | 0 | 41 | 0 | 0 | Unclassified |
| OTU63 | 284 | 154 | 55 | 94 | k_Bacteria; |
| | | | | | p_Proteobacteria; |
| | | | | | c_Alphaproteobacteria; |
| | | | | | o_Rickettsiales; |
| | | | | | f_Pelagibacteraceae; |
| | | | | | g_; |
| | | | | | s_ |
| OTU64 | 18 | 8 | 2 | 0 | Unclassified |
| OTU65 | 0 | 15 | 0 | 7 | k_Bacteria |
| OTU66 | 0 | 0 | 1 | 36 | k_Bacteria |
| OTU67 | 103 | 49 | 17 | 10 | k_Bacteria; |
| | | | | | p_Proteobacteria; |
| | | | | | c_Gammaproteobacteria; |
| | | | | | o_Oceanospirillales; |
| | | | | | f_SUP05; |
| | | | | | g_; |
| | | | | | s_ |
| OTU68 | 30 | 0 | 0 | 0 | Unclassified |
| OTU69 | 0 | 0 | 30 | 0 | Unclassified |
| OTU70 | 105 | 129 | 134 | 434 | k_Bacteria; |
| | | | | | p_Proteobacteria; |
| | | | | | c_Betaproteobacteria; |
| | | | | | o_Burkholderiales; |
| | | | | | f_Comamonadaceae |
| OTU71 | 0 | 0 | 0 | 25 | Unclassified |
| OTU72 | 0 | 0 | 5 | 10 | k_Bacteria |
| OTU73 | 0 | 0 | 0 | 21 | k_Bacteria |
| OTU74 | 30 | 0 | 0 | 0 | Unclassified |
| OTU75 | 28 | 0 | 0 | 0 | Unclassified |
| OTU76 | 10 | 1 | 1 | 0 | k_Bacteria |
| OTU77 | 9 | 8 | 1 | 4 | k_Bacteria |
| OTU78 | 127 | 66 | 31 | 17 | k_Archaea; |
| | | | | | p_Crenarchaeota; |
| | | | | | c_Thaumarchaeota; |
| | | | | | o_Cenarchaeales; |
| | | | | | f_Cenarchaeaceae; |
| | | | | | g_Nitrosopumilus; |
| | | | | | s_ |
| OTU79 | 15 | 3 | 0 | 0 | k_Bacteria |
| OTU80 | 28 | 0 | 0 | 0 | k_Bacteria |
| OTU81 | 32 | 0 | 0 | 0 | k_Bacteria |
| OTU82 | 0 | 5 | 8 | 2 | k_Bacteria |
| OTU83 | 0 | 0 | 21 | 1 | k_Bacteria |
| OTU84 | 0 | 0 | 0 | 24 | Unclassified |
| OTU85 | 11 | 8 | 18 | 9 | k_Bacteria; |
| | | | | | p_Proteobacteria; |
| | | | | | c_Alphaproteobacteria; |
| | | | | | o_Rickettsiales; |
| | | | | | f_mitochondria |
| OTU86 | 27 | 9 | 3 | 6 | k_Bacteria; |
| | | | | | p_Firmicutes; |
| | | | | | c_Bacilli; |
| | | | | | o_Lactobacillales; |
| | | | | | f_Enterococcaceae; |
| | | | | | g_Enterococcus; |
| | | | | | s_ |
| OTU87 | 111 | 43 | 25 | 4 | k_Archaea; |
| | | | | | p_Euryarchaeota; |
| | | | | | c_Thermoplasmata; |
| | | | | | o_E2; |
| | | | | | f_Marine |
| OTU88 | 12 | 13 | 0 | 0 | Unclassified |
| OTU89 | 19 | 0 | 0 | 0 | Unclassified |
| OTU90 | 4 | 0 | 3 | 7 | Unclassified |
| OTU91 | 6 | 10 | 0 | 12 | k_Bacteria |
| OTU92 | 0 | 28 | 0 | 0 | Unclassified |
| OTU93 | 0 | 15 | 4 | 0 | Unclassified |
| OTU94 | 0 | 25 | 0 | 0 | k_Bacteria |
| OTU95 | 0 | 0 | 0 | 44 | k_Bacteria |
| OTU96 | 0 | 0 | 0 | 29 | Unclassified |
| OTU97 | 3 | 4 | 1 | 3 | k_Bacteria |
| OTU98 | 16 | 0 | 0 | 0 | k_Bacteria; |
| | | | | | p_Firmicutes; |
| | | | | | c_Bacilli; |
| | | | | | o_Lactobacillales; |
| | | | | | f_Enterococcaceae; |
| | | | | | g_Enterococcus; |
| | | | | | s_ |
| OTU99 | 4 | 3 | 11 | 0 | k_Bacteria |
| OTU100 | 25 | 7 | 9 | 3 | Unclassified |
| OTU101 | 21 | 15 | 15 | 6 | Unclassified |
| OTU102 | 5 | 6 | 1 | 3 | k_Bacteria; |
| | | | | | p_Proteobacteria |
| OTU103 | 0 | 17 | 2 | 4 | Unclassified |
| OTU104 | 4 | 22 | 16 | 2 | Unclassified |
| OTU105 | 0 | 21 | 0 | 0 | Unclassified |
| OTU106 | 0 | 0 | 16 | 0 | Unclassified |
| OTU107 | 5 | 6 | 2 | 5 | k_Bacteria; |
| | | | | | p_Proteobacteria |
| OTU108 | 6 | 2 | 5 | 4 | k_Bacteria |
| OTU109 | 32 | 0 | 0 | 0 | Unclassified |
| OTU110 | 5 | 5 | 7 | 0 | k_Bacteria |
| OTU111 | 12 | 0 | 5 | 0 | k_Bacteria; |
| | | | | | p_Proteobacteria; |
| | | | | | c_Gammaproteobacteria; |
| | | | | | o_Pseudomonadales; |
| | | | | | f_Pseudomonadaceae |
| OTU112 | 58 | 25 | 6 | 11 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU113 | 5 | 16 | 0 | 0 | Unclassified |
| OTU114 | 7 | 4 | 6 | 3 | k_Bacteria |
| OTU115 | 0 | 3 | 24 | 12 | k_Bacteria |
| OTU116 | 4 | 4 | 3,636 | 2,535 | k_Bacteria; |
| | | | | | p_Firmicutes; |
| | | | | | c_Bacilli; |
| | | | | | o_Bacillales; |
| | | | | | f_Paenibacillaceae; |
| | | | | | g_Paenibacillus; |
| | | | | | s_ |
| OTU117 | 0 | 0 | 0 | 63 | k_Bacteria |
| OTU118 | 0 | 0 | 0 | 54 | k_Bacteria |
| OTU119 | 0 | 0 | 0 | 36 | Unclassified |
| OTU120 | 8 | 2 | 2 | 1 | Unclassified |
| OTU121 | 7 | 1 | 2 | 1 | k_Bacteria |
| OTU122 | 12 | 0 | 0 | 0 | Unclassified |
| OTU123 | 88 | 56 | 25 | 43 | k_Bacteria; |
| | | | | | p_Proteobacteria; |
| | | | | | c_Gammaproteobacteria; |
| | | | | | o_Alteromonadales; |
| | | | | | f_OM60; |
| | | | | | g_; |
| | | | | | s_ |
| OTU124 | 16 | 13 | 0 | 0 | k_Bacteria |
| OTU125 | 10 | 0 | 0 | 0 | k_Bacteria |
| OTU126 | 93 | 52 | 17 | 39 | k_Bacteria; |
| | | | | | p_Proteobacteria; |
| | | | | | c_Gammaproteobacteria; |
| | | | | | o_Oceanospirillales; |
| | | | | | f_Halomonadaceae; |
| | | | | | g_Candidatus |
| OTU127 | 70 | 47 | 15 | 22 | k_Bacteria; |
| | | | | | p_Proteobacteria; |
| | | | | | c_Alphaproteobacteria; |
| | | | | | o_Rhodobacterales; |
| | | | | | f_Rhodobacteraceae; |
| | | | | | g_Octadecabacter; |
| | | | | | s_ |
| OTU128 | 12,016 | 12,027 | 14,707 | 8,742 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU129 | 0 | 16 | 2 | 5 | k_Bacteria; |
| | | | | | p_Proteobacteria; |
| | | | | | c_Gammaproteobacteria; |
| | | | | | o_Xanthomonadales; |
| | | | | | f_Xanthomonadaceae; |
| | | | | | g_Stenotrophomonas; |
| | | | | | s_ |
| OTU130 | 0 | 19 | 2 | 1 | k_Bacteria |
| OTU131 | 0 | 0 | 27 | 2 | k_Bacteria |
| OTU132 | 5 | 3 | 12 | 7 | k_Bacteria |
| OTU133 | 0 | 0 | 4 | 4 | k_Bacteria |
| OTU134 | 0 | 0 | 0 | 31 | Unclassified |
| OTU135 | 2 | 3 | 5 | 0 | Unclassified |
| OTU136 | 12 | 0 | 5 | 0 | k_Bacteria; |
| | | | | | p_Proteobacteria; |
| | | | | | c_Gammaproteobacteria; |
| | | | | | o_Pseudomonadales; |
| | | | | | f_Pseudomonadaceae |
| OTU137 | 16 | 0 | 0 | 0 | k_Bacteria |
| OTU138 | 19 | 0 | 0 | 11 | k_Bacteria; |
| | | | | | p_Proteobacteria |
| OTU139 | 1 | 8 | 4 | 3 | k_Bacteria |
| OTU140 | 0 | 1 | 1,071 | 1,421 | k_Bacteria; |
| | | | | | p_Firmicutes; |
| | | | | | c_Clostridia; |
| | | | | | o_Clostridiales; |
| | | | | | f_Ruminococcaceae; |
| | | | | | g_Clostridium; |
| | | | | | s_hungatei |
| OTU141 | 0 | 0 | 36 | 0 | Unclassified |
| OTU142 | 0 | 0 | 11 | 0 | k_Bacteria |
| OTU143 | 0 | 0 | 0 | 9 | k_Bacteria |
| OTU144 | 6 | 0 | 2 | 0 | k_Bacteria |
| OTU145 | 57 | 54 | 46 | 39 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU146 | 58 | 34 | 14 | 20 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU147 | 61 | 40 | 13 | 73 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_; |
| | | | | | s_ |
| OTU148 | 43 | 12 | 7 | 14 | k_Bacteria |
| OTU149 | 20 | 0 | 0 | 1 | Unclassified |
| OTU150 | 9 | 0 | 0 | 0 | k_Bacteria; |
| | | | | | p_Proteobacteria |
| OTU151 | 30 | 17 | 8 | 1 | k_Bacteria; |
| | | | | | p_Chloroflexi; |
| | | | | | c_SAR202; |
| | | | | | o_; |
| | | | | | f_; |
| | | | | | g_; |
| | | | | | s_ |
| OTU152 | 72 | 77 | 114 | 71 | k_Bacteria; |
| | | | | | p_Cyanobacteria; |
| | | | | | c_Chloroplast; |
| | | | | | o_Streptophyta; |
| | | | | | f_; |
| | | | | | g_; |
| | | | | | s_ |
| OTU153 | 9 | 2 | 1 | 2 | k_Bacteria |
| OTU154 | 0 | 12 | 6 | 20 | Unclassified |
| OTU155 | 0 | 6 | 0 | 0 | k_Bacteria |
| OTU156 | 716 | 546 | 423 | 48 | k_Bacteria; |
| | | | | | p_Firmicutes; |
| | | | | | c_Bacilli; |
| | | | | | o_Lactobacillales; |
| | | | | | f_Leuconostocaceae; |
| | | | | | g_; |
| | | | | | s_ |
| OTU157 | 11 | 18 | 13 | 4 | Unclassified |
| OTU158 | 0 | 13 | 8 | 20 | Unclassified |
| OTU159 | 0 | 8 | 1 | 1 | k_Bacteria; |
| | | | | | p_Proteobacteria; |
| | | | | | c_Gammaproteobacteria; |
| | | | | | o_Xanthomonadales; |
| | | | | | f_Xanthomonadaceae; |
| | | | | | g_Stenotrophomonas; |
| | | | | | s_ |
| OTU160 | 0 | 7 | 2 | 0 | k_Bacteria |
| OTU161 | 0 | 3 | 10 | 11 | k_Bacteria |
| OTU162 | 0 | 0 | 10 | 0 | k_Bacteria |
| OTU163 | 0 | 0 | 0 | 7 | k_Bacteria |
| OTU164 | 0 | 0 | 0 | 24 | Unclassified |
| OTU165 | 56 | 26 | 12 | 19 | k_Bacteria; |
| | | | | | p_Proteobacteria; |
| | | | | | c_Gammaproteobacteria; |
| | | | | | o_Oceanospirillales; |
| | | | | | f_Halomonadaceae; |
| | | | | | g_Candidatus |
| OTU166 | 16 | 6 | 2 | 8 | k_Bacteria; |
| | | | | | p_Proteobacteria; |
| | | | | | c_Alphaproteobacteria |
| OTU167 | 3 | 2 | 3 | 1 | k_Bacteria |
| OTU168 | 8 | 3 | 0 | 0 | k_Bacteria |
| OTU169 | 8 | 3 | 7 | 8 | Unclassified |
| OTU170 | 12 | 0 | 0 | 0 | k_Bacteria |
| OTU171 | 12 | 7 | 2 | 0 | k_Bacteria |
| OTU172 | 6 | 1 | 2 | 1 | k_Bacteria |
| OTU173 | 37 | 19 | 5 | 25 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_Tenacibaculum; |
| | | | | | s_ |
| OTU174 | 6 | 0 | 0 | 0 | Unclassified |
| OTU175 | 11 | 0 | 0 | 0 | k_Bacteria |
| OTU176 | 7 | 0 | 0 | 0 | k_Bacteria |
| OTU177 | 6 | 1 | 1 | 0 | k_Bacteria |
| OTU178 | 55 | 26 | 10 | 16 | k_Bacteria; |
| | | | | | p_Proteobacteria; |
| | | | | | c_Alphaproteobacteria; |
| | | | | | o_Kiloniellales; |
| | | | | | f_; |
| | | | | | g_; |
| | | | | | s_ |
| OTU179 | 12 | 0 | 0 | 0 | k_Bacteria |
| OTU180 | 4 | 1 | 2 | 1 | Unclassified |
| OTU181 | 4 | 7 | 0 | 5 | k_Bacteria |
| OTU182 | 1 | 35 | 5 | 0 | k_Bacteria; |
| | | | | | p_Proteobacteria; |
| | | | | | c_Alphaproteobacteria; |
| | | | | | o_Rickettsiales; |
| | | | | | f_mitochondria |
| OTU183 | 36 | 20 | 10 | 13 | k_Bacteria; |
| | | | | | p_Proteobacteria; |
| | | | | | c_Alphaproteobacteria |
| OTU184 | 0 | 1 | 10 | 7 | k_Bacteria |
| OTU185 | 0 | 0 | 12 | 0 | Unclassified |
| OTU186 | 0 | 0 | 10 | 2 | Unclassified |
| OTU187 | 0 | 0 | 0 | 6 | k_Bacteria |
| OTU188 | 0 | 0 | 0 | 10 | k_Bacteria |
| OTU189 | 0 | 0 | 0 | 57 | k_Bacteria; |
| | | | | | p_Proteobacteria; |
| | | | | | c_Alphaproteobacteria; |
| | | | | | o_Rickettsiales; |
| | | | | | f_mitochondria |
| OTU190 | 1 | 1 | 3 | 12 | k_Bacteria |
| OTU191 | 7 | 3 | 4 | 12 | k_Bacteria; |
| | | | | | p_Proteobacteria; |
| | | | | | c_Gammaproteobacteria; |
| | | | | | o_Enterobacteriales; |
| | | | | | f_Enterobacteriaceae; |
| | | | | | g_Escherichia; |
| | | | | | s_coli |
| OTU192 | 0 | 0 | 1 | 7 | k_Bacteria |
| OTU193 | 0 | 0 | 0 | 16 | k_Bacteria |
| OTU194 | 4 | 0 | 1 | 0 | k_Bacteria |
| OTU195 | 48 | 15 | 4 | 6 | k_Archaea; |
| | | | | | p_Crenarchaeota; |
| | | | | | c_Thaumarchaeota; |
| | | | | | o_Cenarchaeales; |
| | | | | | f_Cenarchaeaceae |
| OTU196 | 45 | 27 | 15 | 13 | k_Bacteria; |
| | | | | | p_Proteobacteria; |
| | | | | | c_Gammaproteobacteria; |
| | | | | | o_Alteromonadales; |
| | | | | | f_OM60; |
| | | | | | g_; |
| | | | | | s_ |
| OTU197 | 4 | 5 | 1 | 2 | k_Bacteria |
| OTU198 | 3 | 2 | 1 | 0 | Unclassified |
| OTU199 | 15 | 20 | 0 | 0 | k_Bacteria |
| OTU200 | 42 | 29 | 9 | 22 | k_Bacteria; |
| | | | | | p_Proteobacteria; |
| | | | | | c_Gammaproteobacteria; |
| | | | | | o_HTCC2188; |
| | | | | | f_HTCC2089; |
| | | | | | g_; |
| | | | | | s_ |
| OTU201 | 3 | 0 | 2 | 0 | Unclassified |
| OTU202 | 17 | 4 | 5 | 1 | k_Bacteria; |
| | | | | | p_Proteobacteria; |
| | | | | | c_Gammaproteobacteria; |
| | | | | | o_Alteromonadales; |
| | | | | | f_Alteromonadaceae; |
| | | | | | g_Glaciecola; |
| | | | | | s_ |
| OTU203 | 5 | 0 | 2 | 0 | Unclassified |
| OTU204 | 8 | 3 | 1 | 2 | k_Bacteria |
| OTU205 | 7 | 1 | 2 | 0 | k_Bacteria |
| OTU206 | 5 | 0 | 4 | 0 | k_Bacteria |
| OTU207 | 5 | 3 | 8 | 3 | Unclassified |
| OTU208 | 37 | 25 | 14 | 5 | k_Bacteria; |
| | | | | | p_Cyanobacteria; |
| | | | | | c_Chloroplast; |
| | | | | | o_Stramenopiles; |
| | | | | | f_; |
| | | | | | g_; |
| | | | | | s_ |
| OTU209 | 2 | 0 | 2 | 1 | k_Bacteria |
| OTU210 | 5 | 0 | 0 | 0 | Unclassified |
| OTU211 | 9 | 5 | 1 | 0 | k_Bacteria; |
| | | | | | p_Proteobacteria; |
| | | | | | c_Alphaproteobacteria |
| OTU212 | 954 | 1,013 | 1,097 | 691 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU213 | 10 | 8 | 5 | 18 | k_Bacteria; |
| | | | | | p_Proteobacteria; |
| | | | | | c_Gammaproteobacteria |
| OTU214 | 33 | 10 | 8 | 15 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_; |
| | | | | | s_ |
| OTU215 | 8 | 4 | 1 | 0 | k_Bacteria |
| OTU216 | 5 | 1 | 5 | 1 | k_Bacteria |
| OTU217 | 1 | 3 | 0 | 4 | k_Bacteria; |
| | | | | | p_Actinobacteria; |
| | | | | | c_Actinobacteria; |
| | | | | | o_Actinomycetales; |
| | | | | | f_Micrococcaceae; |
| | | | | | g_Rothia |
| OTU218 | 3 | 8 | 1 | 1 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU219 | 9 | 12 | 40 | 42 | k_Bacteria; |
| | | | | | p_Firmicutes; |
| | | | | | c_Bacilli; |
| | | | | | o_Bacillales; |
| | | | | | f_Bacillaceae; |
| | | | | | g_Bacillus; |
| | | | | | s_ |
| OTU220 | 217 | 236 | 74 | 149 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU221 | 0 | 3 | 0 | 4 | Unclassified |
| OTU222 | 0 | 3 | 0 | 2 | k_Bacteria; |
| | | | | | p_Actinobacteria; |
| | | | | | c_Actinobacteria; |
| | | | | | o_Actinomycetales; |
| | | | | | f_Micrococcaceae; |
| | | | | | g_Rothia |
| OTU223 | 1,431 | 1,360 | 1,413 | 1,007 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU224 | 7 | 3 | 10 | 4 | k_Bacteria; |
| | | | | | p_Proteobacteria |
| OTU225 | 0 | 6 | 0 | 0 | Unclassified |
| OTU226 | 0 | 0 | 4 | 6 | Unclassified |
| OTU227 | 0 | 0 | 12 | 0 | Unclassified |
| OTU228 | 0 | 1 | 26 | 11 | k_Bacteria |
| OTU229 | 1 | 0 | 11 | 2 | Unclassified |
| OTU230 | 0 | 0 | 0 | 12 | k_Bacteria |
| OTU231 | 0 | 0 | 0 | 6 | k_Bacteria |
| OTU232 | 0 | 0 | 71 | 43 | k_Bacteria; |
| | | | | | p_Firmicutes; |
| | | | | | c_Clostridia; |
| | | | | | o_Clostridiales; |
| | | | | | f_Peptostreptococcaceae; |
| | | | | | g_Clostridium; |
| | | | | | s_venationis |
| OTU233 | 0 | 0 | 2 | 7 | k_Bacteria |
| OTU234 | 0 | 0 | 0 | 5 | k_Bacteria |
| OTU235 | 0 | 0 | 0 | 5 | k_Bacteria |
| OTU236 | 12 | 7 | 3 | 6 | k_Bacteria; |
| | | | | | p_Bacteroidetes; |
| | | | | | c_Sphingobacteriia; |
| | | | | | o_Sphingobacteriales; |
| | | | | | f_NS11-12; |
| | | | | | g_; |
| | | | | | s_ |
| OTU237 | 3 | 5 | 4 | 0 | k_Bacteria |
| OTU238 | 5 | 0 | 0 | 0 | Unclassified |
| OTU239 | 10 | 5 | 2 | 4 | k_Bacteria; |
| | | | | | p_Proteobacteria; |
| | | | | | c_Gammaproteobacteria |
| OTU240 | 4 | 0 | 5 | 3 | k_Bacteria; |
| | | | | | p_Proteobacteria; |
| | | | | | c_Alphaproteobacteria; |
| | | | | | o_Rickettsiales; |
| | | | | | f_mitochondria |
| OTU241 | 17 | 6 | 3 | 1 | k_Bacteria |
| OTU242 | 19 | 7 | 4 | 12 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_; |
| | | | | | s_ |
| OTU243 | 6 | 0 | 0 | 0 | Unclassified |
| OTU244 | 12 | 0 | 0 | 0 | k_Bacteria |
| OTU245 | 4 | 2 | 1 | 2 | k_Bacteria |
| OTU246 | 2 | 0 | 1 | 0 | k_Bacteria |
| OTU247 | 5 | 1 | 3 | 0 | Unclassified |
| OTU248 | 5 | 1 | 0 | 0 | k_Bacteria |
| OTU249 | 5 | 0 | 3 | 0 | k_Bacteria |
| OTU250 | 12 | 8 | 3 | 2 | k_Bacteria; |
| | | | | | p_Bacteroidetes; |
| | | | | | c_Flavobacteriia; |
| | | | | | o_Flavobacteriales |
| OTU251 | 30 | 13 | 4 | 11 | k_Bacteria; |
| | | | | | p_Proteobacteria; |
| | | | | | c_Alphaproteobacteria; |
| | | | | | o_Rickettsiales; |
| | | | | | f_Pelagibacteraceae; |
| | | | | | g_; |
| | | | | | s_ |
| OTU252 | 3 | 0 | 1 | 0 | k_Bacteria |
| OTU253 | 52 | 35 | 8 | 21 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU254 | 4 | 0 | 0 | 0 | k_Bacteria |
| OTU255 | 5 | 0 | 0 | 0 | k_Bacteria |
| OTU256 | 21 | 11 | 8 | 3 | k_Archaea; |
| | | | | | p_Euryarchaeota; |
| | | | | | c_Thermoplasmata; |
| | | | | | o_E2; |
| | | | | | f_Marine |
| OTU257 | 602 | 381 | 201 | 14 | k_Bacteria; |
| | | | | | p_Firmicutes; |
| | | | | | c_Bacilli; |
| | | | | | o_Lactobacillales; |
| | | | | | f_Lactobacillaceae; |
| | | | | | g_Pediococcus; |
| | | | | | s_ |
| OTU258 | 21 | 14 | 5 | 1 | k_Bacteria |
| OTU259 | 3 | 1 | 0 | 0 | k_Bacteria |
| OTU260 | 43 | 55 | 56 | 55 | k_Bacteria; |
| | | | | | p_Proteobacteria; |
| | | | | | c_Gammaproteobacteria; |
| | | | | | o_Enterobacteriales; |
| | | | | | f_Enterobacteriaceae; |
| | | | | | g_Pantoea |
| OTU261 | 3 | 0 | 2 | 0 | k_Bacteria |
| OTU262 | 18 | 5 | 0 | 6 | k_Bacteria; |
| | | | | | p_Proteobacteria; |
| | | | | | c_Gammaproteobacteria; |
| | | | | | o_Thiotrichales; |
| | | | | | f_Piscirickettsiaceae; |
| | | | | | g_Thiomicrospira; |
| | | | | | s_frisia |
| OTU263 | 5 | 0 | 0 | 0 | Unclassified |
| OTU264 | 5 | 0 | 0 | 0 | k_Bacteria |
| OTU265 | 3 | 0 | 1 | 0 | Unclassified |
| OTU266 | 7 | 0 | 0 | 0 | Unclassified |
| OTU267 | 4 | 0 | 0 | 0 | k_Bacteria; |
| | | | | | p_Bacteroidetes; |
| | | | | | c_Flavobacteriia; |
| | | | | | o_Flavobacteriales |
| OTU268 | 19 | 14 | 1 | 5 | k_Bacteria; |
| | | | | | p_Bacteroidetes; |
| | | | | | c_Flavobacteriia; |
| | | | | | o_Flavobacteriales; |
| | | | | | f_Cryomorphaceae; |
| | | | | | g_Fluviicola; |
| | | | | | s_ |
| OTU269 | 12 | 21 | 5 | 17 | Unclassified |
| OTU270 | 36 | 19 | 5 | 30 | k_Bacteria; |
| | | | | | p_Proteobacteria |
| OTU271 | 42 | 14 | 6 | 4 | k_Bacteria; |
| | | | | | p_Cyanobacteria; |
| | | | | | c_Chloroplast; |
| | | | | | o_Cryptophyta; |
| | | | | | f_; |
| | | | | | g_; |
| | | | | | s_ |
| OTU272 | 6 | 0 | 0 | 0 | k_Bacteria |
| OTU273 | 18 | 0 | 0 | 12 | k_Bacteria; |
| | | | | | p_Proteobacteria; |
| | | | | | c_Alphaproteobacteria; |
| | | | | | o_Rickettsiales; |
| | | | | | f_mitochondria |
| OTU274 | 17 | 7 | 4 | 11 | k_Bacteria; |
| | | | | | p_Proteobacteria; |
| | | | | | c_Alphaproteobacteria |
| OTU275 | 16 | 10 | 1 | 4 | k_Bacteria; |
| | | | | | p_Proteobacteria; |
| | | | | | c_Gammaproteobacteria; |
| | | | | | o_Vibrionales; |
| | | | | | f_Pseudoalteromonadaceae; |
| | | | | | g_Pseudoalteromonas |
| OTU276 | 10 | 4 | 0 | 8 | k_Bacteria; |
| | | | | | p_Bacteroidetes |
| OTU277 | 2 | 1 | 0 | 0 | Unclassified |
| OTU278 | 3 | 6 | 1 | 4 | k_Bacteria; |
| | | | | | p_Proteobacteria; |
| | | | | | c_Alphaproteobacteria; |
| | | | | | o_Sphingomonadales; |
| | | | | | f_Sphingomonadaceae; |
| | | | | | g_Sphingomonas |
| OTU279 | 0 | 35 | 16 | 1 | k_Bacteria |
| OTU280 | 23 | 19 | 6 | 6 | k_Bacteria; |
| | | | | | p_Fusobacteria; |
| | | | | | c_Fusobacteriia; |
| | | | | | o_Fusobacteriales; |
| | | | | | f_Fusobacteriaceae; |
| | | | | | g_Propionigenium; |
| | | | | | s_ |
| OTU281 | 486 | 470 | 299 | 274 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU282 | 2 | 5 | 3 | 0 | k_Bacteria; |
| | | | | | p_Bacteroidetes; |
| | | | | | c_Flavobacteriia; |
| | | | | | o_Flavobacteriales; |
| | | | | | f_[Weeksellaceae]; |
| | | | | | g_Chryseobacterium; |
| | | | | | s_ |
| OTU283 | 8 | 20 | 5 | 22 | Unclassified |
| OTU284 | 16 | 12 | 8 | 2 | k_Bacteria; |
| | | | | | p_Proteobacteria |
| OTU285 | 30 | 21 | 4 | 10 | k_Bacteria |
| OTU286 | 0 | 3 | 0 | 0 | Unclassified |
| OTU287 | 15 | 17 | 7 | 10 | k_Bacteria; |
| | | | | | p_Proteobacteria; |
| | | | | | c_Betaproteobacteria; |
| | | | | | o_Methylophilales |
| OTU288 | 0 | 7 | 0 | 0 | k_Bacteria; |
| | | | | | p_Proteobacteria; |
| | | | | | c_Alphaproteobacteria; |
| | | | | | o_Rickettsiales; |
| | | | | | f_mitochondria |
| OTU289 | 0 | 2 | 1 | 0 | k_Bacteria |
| OTU290 | 4 | 3 | 1 | 0 | k_Bacteria |
| OTU291 | 0 | 9 | 0 | 1 | k_Bacteria; |
| | | | | | p_Proteobacteria |
| OTU292 | 0 | 0 | 3 | 1 | k_Bacteria |
| OTU293 | 0 | 0 | 2 | 2 | k_Bacteria |
| OTU294 | 51 | 43 | 33 | 21 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU295 | 56,288 | 55,419 | 61,272 | 57,301 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU296 | 0 | 0 | 143 | 293 | k_Bacteria; |
| | | | | | p_Firmicutes; |
| | | | | | c_Clostridia; |
| | | | | | o_Clostridiales; |
| | | | | | f_Lachnospiraceae; |
| | | | | | g_Dorea; |
| | | | | | s_ |
| OTU297 | 0 | 0 | 5 | 3 | Unclassified |
| OTU298 | 1 | 2 | 3 | 2 | k_Bacteria |
| OTU299 | 1 | 1 | 15 | 5 | k_Bacteria; |
| | | | | | p_Proteobacteria; |
| | | | | | c_Gammaproteobacteria; |
| | | | | | o_Pasteurellales; |
| | | | | | f_Pasteurellaceae; |
| | | | | | g_Haemophilus; |
| | | | | | s_parainfluenzae |
| OTU300 | 7 | 5 | 7 | 9 | k_Bacteria |
| OTU301 | 0 | 0 | 4 | 5 | k_Bacteria |
| OTU302 | 0 | 0 | 7 | 0 | Unclassified |
| OTU303 | 0 | 0 | 0 | 5 | Unclassified |
| OTU304 | 4 | 10 | 16 | 56 | k_Bacteria; |
| | | | | | p_Firmicutes; |
| | | | | | c_Clostridia; |
| | | | | | o_Clostridiales; |
| | | | | | f_Peptostreptococcaceae; |
| | | | | | g_Peptostreptococcus; |
| | | | | | s_anaerobius |
| OTU305 | 0 | 0 | 0 | 35 | k_Bacteria |
| OTU306 | 0 | 0 | 0 | 10 | k_Bacteria; |
| | | | | | p_Bacteroidetes; |
| | | | | | c_Flavobacteriia; |
| | | | | | o_Flavobacteriales; |
| | | | | | f_Flavobacteriaceae |
| OTU307 | 1 | 0 | 0 | 4 | k_Bacteria; |
| | | | | | p_Proteobacteria; |
| | | | | | c_Deltaproteobacteria; |
| | | | | | o_Myxococcales; |
| | | | | | f_Nannocystaceae; |
| | | | | | g_Plesiocystis; |
| | | | | | s_ |
| OTU308 | 0 | 0 | 0 | 17 | k_Bacteria |
| OTU309 | 0 | 0 | 0 | 4 | k_Bacteria; |
| | | | | | p_Proteobacteria |
| OTU310 | 0 | 0 | 1 | 7 | k_Bacteria |
| OTU311 | 0 | 0 | 0 | 8 | Unclassified |
| OTU312 | 6 | 5 | 1 | 5 | k_Bacteria; |
| | | | | | p_Proteobacteria; |
| | | | | | c_Betaproteobacteria; |
| | | | | | o_Methylophilales; |
| | | | | | f_Methylophilaceae; |
| | | | | | g_; |
| | | | | | s_ |
| OTU313 | 134 | 58 | 18 | 51 | k_Bacteria; |
| | | | | | p_Cyanobacteria; |
| | | | | | c_Synechococcophycideae; |
| | | | | | o_Synechococcales; |
| | | | | | f_Synechococcaceae |
| OTU314 | 8 | 5 | 2 | 3 | k_Bacteria; |
| | | | | | p_Proteobacteria; |
| | | | | | c_Gammaproteobacteria; |
| | | | | | o_Alteromonadales; |
| | | | | | f_HTCC2188; |
| | | | | | g_HTCC; |
| | | | | | s_ |
| OTU315 | 3 | 0 | 0 | 0 | k_Bacteria; |
| | | | | | p_Planctomycetes; |
| | | | | | c_Phycisphaerae; |
| | | | | | o_Phycisphaerales; |
| | | | | | f_; |
| | | | | | g_; |
| | | | | | s_ |
| OTU316 | 3 | 0 | 0 | 1 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_; |
| | | | | | s_ |
| OTU317 | 64 | 30 | 20 | 17 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU318 | 3 | 0 | 0 | 0 | k_Bacteria; |
| | | | | | p_Proteobacteria; |
| | | | | | c_Deltaproteobacteria; |
| | | | | | o_Myxococcales; |
| | | | | | f_OM27; |
| | | | | | g_; |
| | | | | | s_ |
| OTU319 | 3 | 0 | 0 | 0 | Unclassified |
| OTU320 | 24 | 6 | 7 | 18 | k_Bacteria; |
| | | | | | p_Proteobacteria; |
| | | | | | c_Gammaproteobacteria; |
| | | | | | o_Alteromonadales; |
| | | | | | f_OM60; |
| | | | | | g_; |
| | | | | | s_ |
| OTU321 | 148 | 102 | 50 | 40 | k_Bacteria; |
| | | | | | p_Firmicutes; |
| | | | | | c_Bacilli; |
| | | | | | o_Lactobacillales; |
| | | | | | f_Lactobacillaceae; |
| | | | | | g_; |
| | | | | | s_ |
| OTU322 | 4 | 3 | 1 | 0 | k_Bacteria; |
| | | | | | p_GN02; |
| | | | | | c_; |
| | | | | | o_; |
| | | | | | f_; |
| | | | | | g_; |
| | | | | | s_ |
| OTU323 | 8 | 0 | 0 | 0 | k_Bacteria |
| OTU324 | 3 | 0 | 0 | 0 | k_Bacteria; |
| | | | | | p_Proteobacteria; |
| | | | | | c_Gammaproteobacteria |
| OTU325 | 2 | 2 | 0 | 0 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_; |
| | | | | | s_ |
| OTU326 | 71 | 55 | 23 | 3 | k_Bacteria; |
| | | | | | p_Firmicutes; |
| | | | | | c_Bacilli; |
| | | | | | o_Lactobacillales; |
| | | | | | f_; |
| | | | | | g_; |
| | | | | | s_ |
| OTU327 | 3 | 4 | 1 | 20 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU328 | 15 | 9 | 4 | 2 | k_Bacteria |
| OTU329 | 3 | 0 | 0 | 0 | k_Bacteria; |
| | | | | | p_Proteobacteria |
| OTU330 | 3 | 0 | 0 | 0 | k_Bacteria; |
| | | | | | p_Bacteroidetes; |
| | | | | | c_Sphingobacteriia; |
| | | | | | o_Sphingobacteriales; |
| | | | | | f_NS11-12; |
| | | | | | g_; |
| | | | | | s_ |
| OTU331 | 6 | 0 | 1 | 0 | k_Bacteria; |
| | | | | | p_Verrucomicrobia; |
| | | | | | c_Verrucomicrobiae; |
| | | | | | o_Verrucomicrobiales; |
| | | | | | f_Verrucomicrobiaceae |
| OTU332 | 5 | 0 | 0 | 0 | k_Bacteria |
| OTU333 | 3 | 1 | 0 | 0 | k_Bacteria; |
| | | | | | p_Chloroflexi; |
| | | | | | c_SAR202; |
| | | | | | o_; |
| | | | | | f_; |
| | | | | | g_; |
| | | | | | s_ |
| OTU334 | 4 | 1 | 1 | 0 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_; |
| | | | | | s_ |
| OTU335 | 6 | 1 | 1 | 1 | k_Bacteria; |
| | | | | | p_Proteobacteria; |
| | | | | | c_Deltaproteobacteria; |
| | | | | | o_Myxococcales; |
| | | | | | f_; |
| | | | | | g_; |
| | | | | | s_ |
| OTU336 | 4 | 1 | 0 | 0 | k_Bacteria |
| OTU337 | 8 | 4 | 1 | 2 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_Saprospira; |
| | | | | | s_ |
| OTU338 | 3 | 0 | 1 | 0 | k_Bacteria |
| OTU339 | 5 | 1 | 0 | 0 | k_Bacteria |
| OTU340 | 108 | 62 | 50 | 3 | k_Bacteria; |
| | | | | | p_Firmicutes; |
| | | | | | c_Bacilli; |
| | | | | | o_Lactobacillales; |
| | | | | | f_Lactobacillaceae; |
| | | | | | g_Lactobacillus; |
| | | | | | s_brevis |
| OTU341 | 3 | 0 | 0 | 0 | Unclassified |
| OTU342 | 5 | 0 | 0 | 0 | k_Bacteria |
| OTU343 | 5 | 1 | 0 | 1 | k_Bacteria; |
| | | | | | p_Chloroflexi; |
| | | | | | c_Anaerolineae; |
| | | | | | o_Caldilineales; |
| | | | | | f_Caldilineaceae |
| OTU344 | 10 | 6 | 3 | 5 | k_Bacteria; |
| | | | | | p_Proteobacteria; |
| | | | | | c_Betaproteobacteria; |
| | | | | | o_Burkholderiales; |
| | | | | | f_Oxalobacteraceae; |
| | | | | | g_Janthinobacterium; |
| | | | | | s_ |
| OTU345 | 3 | 2 | 0 | 0 | k_Bacteria; |
| | | | | | p_Fusobacteria; |
| | | | | | c_Fusobacteriia; |
| | | | | | o_Fusobacteriales; |
| | | | | | f_Fusobacteriaceae; |
| | | | | | g_Propionigenium; |
| | | | | | s_ |
| OTU346 | 6 | 1 | 0 | 0 | k_Bacteria; |
| | | | | | p_Planctomycetes; |
| | | | | | c_Planctomycetia; |
| | | | | | o_Pirellulales; |
| | | | | | f_Pirellulaceae; |
| | | | | | g_; |
| | | | | | s_ |
| OTU347 | 5 | 2 | 5 | 0 | k_Bacteria; |
| | | | | | p_Proteobacteria; |
| | | | | | c_Gammaproteobacteria; |
| | | | | | o_Pseudomonadales; |
| | | | | | f_Moraxellaceae; |
| | | | | | g_Acinetobacter; |
| | | | | | s_rhizosphaerae |
| OTU348 | 2 | 0 | 0 | 0 | k_Bacteria; |
| | | | | | p_Verrucomicrobia; |
| | | | | | c_Verrucomicrobiae; |
| | | | | | o_Verrucomicrobiales; |
| | | | | | f_Verrucomicrobiaceae |
| OTU349 | 6 | 4 | 7 | 5 | k_Bacteria |
| OTU350 | 4 | 1 | 1 | 0 | k_Bacteria |
| OTU351 | 7 | 2 | 1 | 0 | k_Bacteria; |
| | | | | | p_Proteobacteria; |
| | | | | | c_Gammaproteobacteria; |
| | | | | | o_Oceanospirillales; |
| | | | | | f_Oceanospirillaceae; |
| | | | | | g_Oleibacter; |
| | | | | | s_ |
| OTU352 | 8 | 7 | 2 | 4 | k_Bacteria; |
| | | | | | p_Proteobacteria; |
| | | | | | c_Gammaproteobacteria; |
| | | | | | o_Thiohalorhabdales; |
| | | | | | f_Thiohalorhabdaceae; |
| | | | | | g_; |
| | | | | | s_ |
| OTU353 | 8 | 4 | 1 | 4 | k_Bacteria; |
| | | | | | p_Proteobacteria; |
| | | | | | c_Alphaproteobacteria; |
| | | | | | o_Rhizobiales; |
| | | | | | f_Hyphomicrobiaceae |
| OTU354 | 17 | 7 | 3 | 1 | k_Bacteria; |
| | | | | | p_Bacteroidetes; |
| | | | | | c_Flavobacteriia; |
| | | | | | o_Flavobacteriales; |
| | | | | | f_Flavobacteriaceae |
| OTU355 | 6 | 1 | 1 | 0 | k_Bacteria; |
| | | | | | p_Actinobacteria; |
| | | | | | c_Acidimicrobiia; |
| | | | | | o_Acidimicrobiales; |
| | | | | | f_OCS155; |
| | | | | | g_; |
| | | | | | s_ |
| OTU356 | 7 | 2 | 1 | 4 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_; |
| | | | | | s_ |
| OTU357 | 3 | 3 | 0 | 0 | k_Bacteria; |
| | | | | | p_Verrucomicrobia; |
| | | | | | c_Opitutae; |
| | | | | | o_Puniceicoccales; |
| | | | | | f_Puniceicoccaceae; |
| | | | | | g_MB11C04; |
| | | | | | s_ |
| OTU358 | 3 | 0 | 0 | 0 | Unclassified |
| OTU359 | 2 | 1 | 1 | 1 | k_Bacteria; |
| | | | | | p_Bacteroidetes; |
| | | | | | c_Flavobacteriia; |
| | | | | | o_Flavobacteriales; |
| | | | | | f_Cryomorphaceae; |
| | | | | | g_; |
| | | | | | s_ |
| OTU360 | 9 | 5 | 1 | 0 | k_Bacteria; |
| | | | | | p_Planctomycetes; |
| | | | | | c_Planctomycetia; |
| | | | | | o_Planctomycetales; |
| | | | | | f_Planctomycetaceae; |
| | | | | | g_Planctomyces; |
| | | | | | s_ |
| OTU361 | 3 | 0 | 0 | 0 | k_Bacteria; |
| | | | | | p_Proteobacteria |
| OTU362 | 4 | 1 | 1 | 0 | k_Bacteria |
| OTU363 | 2 | 0 | 1 | 0 | k_Bacteria |
| OTU364 | 16 | 9 | 4 | 7 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_; |
| | | | | | s_ |
| OTU365 | 3 | 0 | 0 | 0 | k_Bacteria |
| OTU366 | 4 | 0 | 2 | 1 | k_Bacteria; |
| | | | | | p_Proteobacteria; |
| | | | | | c_Gammaproteobacteria; |
| | | | | | o_Alteromonadales; |
| | | | | | f_Moritellaceae; |
| | | | | | g_Moritella; |
| | | | | | s_ |
| OTU367 | 7 | 2 | 1 | 2 | k_Bacteria; |
| | | | | | p_Proteobacteria; |
| | | | | | c_Alphaproteobacteria; |
| | | | | | o_Rickettsiales; |
| | | | | | f_mitochondria |
| OTU368 | 3 | 0 | 0 | 0 | Unclassified |
| OTU369 | 14 | 3 | 1 | 1 | k_Bacteria; |
| | | | | | p_Verrucomicrobia; |
| | | | | | c_Verrucomicrobiae; |
| | | | | | o_Verrucomicrobiales; |
| | | | | | f_Verrucomicrobiaceae |
| OTU370 | 3 | 0 | 0 | 1 | k_Bacteria; |
| | | | | | p_Proteobacteria; |
| | | | | | c_Betaproteobacteria; |
| | | | | | o_Burkholderiales; |
| | | | | | f_Comamonadaceae |
| OTU371 | 3 | 4 | 0 | 0 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_Saprospira; |
| | | | | | s_ |
| OTU372 | 2 | 3 | 0 | 0 | Unclassified |
| OTU373 | 3 | 0 | 0 | 0 | k_Bacteria |
| OTU374 | 3 | 0 | 0 | 0 | k_Bacteria |
| OTU375 | 6 | 0 | 0 | 2 | k_Bacteria |
| OTU376 | 5 | 3 | 1 | 2 | k_Bacteria |
| OTU377 | 6 | 0 | 1 | 0 | k_Bacteria; |
| | | | | | p_Proteobacteria; |
| | | | | | c_Alphaproteobacteria; |
| | | | | | o_Rhodobacterales; |
| | | | | | f_Rhodobacteraceae; |
| | | | | | g_; |
| | | | | | s_ |
| OTU378 | 5 | 0 | 0 | 0 | Unclassified |
| OTU379 | 4 | 0 | 1 | 0 | k_Bacteria; |
| | | | | | p_Proteobacteria; |
| | | | | | c_Alphaproteobacteria; |
| | | | | | o_Rhodobacterales; |
| | | | | | f_Rhodobacteraceae; |
| | | | | | g_Rhodobacter; |
| | | | | | s_ |
| OTU380 | 3 | 0 | 0 | 0 | k_Bacteria; |
| | | | | | p_Proteobacteria; |
| | | | | | c_Deltaproteobacteria; |
| | | | | | o_Myxococcales; |
| | | | | | f_OM27; |
| | | | | | g_; |
| | | | | | s_ |
| OTU381 | 4 | 0 | 1 | 0 | k_Bacteria; |
| | | | | | p_Actinobacteria; |
| | | | | | c_Acidimicrobiia; |
| | | | | | o_Acidimicrobiales; |
| | | | | | f_OCS155; |
| | | | | | g_; |
| | | | | | s_ |
| OTU382 | 14 | 2 | 0 | 6 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU383 | 4 | 0 | 0 | 0 | k_Bacteria |
| OTU384 | 16 | 8 | 22 | 6 | k_Bacteria; |
| | | | | | p_Firmicutes; |
| | | | | | c_Bacilli; |
| | | | | | o_Lactobacillales; |
| | | | | | f_Leuconostocaceae; |
| | | | | | g_Leuconostoc; |
| | | | | | s_ |
| OTU385 | 4 | 4 | 0 | 0 | Unclassified |
| OTU386 | 20 | 3 | 1 | 5 | k_Bacteria; |
| | | | | | p_Proteobacteria; |
| | | | | | c_Alphaproteobacteria |
| OTU387 | 3 | 0 | 0 | 0 | k_Bacteria; |
| | | | | | p_Bacteroidetes |
| OTU388 | 12 | 6 | 1 | 1 | k_Archaea; |
| | | | | | p_Euryarchaeota; |
| | | | | | c_Thermoplasmata; |
| | | | | | o_E2; |
| | | | | | f_Marine |
| OTU389 | 3 | 0 | 1 | 0 | k_Bacteria |
| OTU390 | 3 | 1 | 0 | 0 | k_Bacteria |
| OTU391 | 7 | 3 | 7 | 2 | k_Bacteria |
| OTU392 | 4 | 3 | 0 | 0 | Unclassified |
| OTU393 | 3 | 0 | 0 | 0 | k_Bacteria; |
| | | | | | p_Proteobacteria; |
| | | | | | c_Alphaproteobacteria |
| OTU394 | 3 | 1 | 0 | 0 | k_Bacteria |
| OTU395 | 4 | 0 | 0 | 0 | Unclassified |
| OTU396 | 6 | 6 | 2 | 2 | k_Bacteria; |
| | | | | | p_Proteobacteria; |
| | | | | | c_Alphaproteobacteria; |
| | | | | | o_Rickettsiales; |
| | | | | | f_; |
| | | | | | g_; |
| | | | | | s_ |
| OTU397 | 3 | 1 | 0 | 0 | k_Bacteria |
| OTU398 | 2 | 0 | 1 | 0 | k_Bacteria |
| OTU399 | 2 | 0 | 0 | 1 | Unclassified |
| OTU400 | 5 | 2 | 1 | 0 | k_Bacteria; |
| | | | | | p_Proteobacteria; |
| | | | | | c_Gammaproteobacteria |
| OTU401 | 34 | 14 | 5 | 2 | k_Bacteria; |
| | | | | | p_Cyanobacteria; |
| | | | | | c_Chloroplast; |
| | | | | | o_Stramenopiles; |
| | | | | | f_; |
| | | | | | g_; |
| | | | | | s_ |
| OTU402 | 3 | 0 | 0 | 0 | Unclassified |
| OTU403 | 3 | 0 | 0 | 0 | k_Bacteria |
| OTU404 | 3 | 0 | 0 | 0 | k_Bacteria |
| OTU405 | 8 | 3 | 1 | 2 | k_Bacteria; |
| | | | | | p_Cyanobacteria; |
| | | | | | c_Chloroplast; |
| | | | | | o_Stramenopiles; |
| | | | | | f_; |
| | | | | | g_; |
| | | | | | s_ |
| OTU406 | 27 | 6 | 8 | 15 | k_Bacteria; |
| | | | | | p_Proteobacteria; |
| | | | | | c_Alphaproteobacteria |
| OTU407 | 4 | 2 | 2 | 0 | k_Bacteria |
| OTU408 | 2 | 1 | 0 | 0 | Unclassified |
| OTU409 | 10 | 4 | 0 | 1 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU410 | 5 | 3 | 0 | 5 | k_Bacteria; |
| | | | | | p_Proteobacteria; |
| | | | | | c_Alphaproteobacteria; |
| | | | | | o_Rhodobacterales; |
| | | | | | f_Rhodobacteraceae; |
| | | | | | g_Loktanella; |
| | | | | | s_ |
| OTU411 | 2 | 1 | 0 | 1 | k_Bacteria |
| OTU412 | 14 | 4 | 1 | 2 | k_Bacteria; |
| | | | | | p_Bacteroidetes; |
| | | | | | c_Flavobacteriia; |
| | | | | | o_Flavobacteriales; |
| | | | | | f_Flavobacteriaceae |
| OTU413 | 3 | 0 | 0 | 0 | k_Bacteria; |
| | | | | | p_Planctomycetes; |
| | | | | | c_Phycisphaerae; |
| | | | | | o_Phycisphaerales; |
| | | | | | f_; |
| | | | | | g_; |
| | | | | | s_ |
| OTU414 | 8 | 6 | 1 | 7 | k_Bacteria; |
| | | | | | p_Bacteroidetes; |
| | | | | | c_Flavobacteriia; |
| | | | | | o_Flavobacteriales; |
| | | | | | f_Flavobacteriaceae |
| OTU415 | 8 | 1 | 0 | 0 | k_Archaea; |
| | | | | | p_Euryarchaeota; |
| | | | | | c_Thermoplasmata; |
| | | | | | o_E2; |
| | | | | | f_Marine |
| OTU416 | 3 | 0 | 0 | 0 | Unclassified |
| OTU417 | 4 | 1 | 0 | 4 | k_Bacteria; |
| | | | | | p_Bacteroidetes; |
| | | | | | c_Flavobacteriia; |
| | | | | | o_Flavobacteriales; |
| | | | | | f_Flavobacteriaceae |
| OTU418 | 6 | 5 | 1 | 1 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU419 | 2 | 3 | 1 | 0 | k_Bacteria |
| OTU420 | 18 | 5 | 1 | 6 | k_Bacteria; |
| | | | | | p_Planctomycetes; |
| | | | | | c_Planctomycetia; |
| | | | | | o_Pirellulales; |
| | | | | | f_Pirellulaceae; |
| | | | | | g_Planctomycete; |
| | | | | | s_LF1 |
| OTU421 | 7 | 3 | 2 | 0 | k_Bacteria; |
| | | | | | p_Proteobacteria; |
| | | | | | c_Alphaproteobacteria |
| OTU422 | 3 | 2 | 1 | 2 | k_Bacteria; |
| | | | | | p_Proteobacteria; |
| | | | | | c_Gammaproteobacteria; |
| | | | | | o_Alteromonadales; |
| | | | | | f_OM60; |
| | | | | | g_; |
| | | | | | s_ |
| OTU423 | 14 | 15 | 29 | 11 | k_Bacteria; |
| | | | | | p_Proteobacteria; |
| | | | | | c_Alphaproteobacteria; |
| | | | | | o_Rickettsiales; |
| | | | | | f_mitochondria |
| OTU424 | 3 | 0 | 0 | 0 | Unclassified |
| OTU425 | 4 | 0 | 0 | 1 | k_Bacteria; |
| | | | | | p_Proteobacteria; |
| | | | | | c_Alphaproteobacteria; |
| | | | | | o_Rhodospirillales; |
| | | | | | f_; |
| | | | | | g_; |
| | | | | | s_ |
| OTU426 | 3 | 0 | 0 | 0 | k_Bacteria |
| OTU427 | 58 | 35 | 17 | 3 | k_Bacteria; |
| | | | | | p_Firmicutes; |
| | | | | | c_Bacilli; |
| | | | | | o_Lactobacillales |
| OTU428 | 6 | 0 | 0 | 0 | k_Bacteria |
| OTU429 | 10 | 2 | 1 | 4 | k_Bacteria; |
| | | | | | p_Proteobacteria; |
| | | | | | c_Gammaproteobacteria; |
| | | | | | o_Oceanospirillales; |
| | | | | | f_Halomonadaceae; |
| | | | | | g_Candidatus |
| OTU430 | 2 | 3 | 0 | 0 | k_Bacteria; |
| | | | | | p_Verrucomicrobia; |
| | | | | | c_Verrucomicrobiae; |
| | | | | | o_Verrucomicrobiales; |
| | | | | | f_Verrucomicrobiaceae; |
| | | | | | g_; |
| | | | | | s_ |
| OTU431 | 12 | 3 | 2 | 3 | k_Bacteria; |
| | | | | | p_Proteobacteria; |
| | | | | | c_Gammaproteobacteria |
| OTU432 | 3 | 0 | 0 | 0 | Unclassified |
| OTU433 | 4 | 4 | 4 | 5 | k_Bacteria; |
| | | | | | p_Planctomycetes; |
| | | | | | c_Planctomycetia; |
| | | | | | o_Pirellulales; |
| | | | | | f_Pirellulaceae; |
| | | | | | g_; |
| | | | | | s_ |
| OTU434 | 5 | 0 | 0 | 0 | k_Bacteria |
| OTU435 | 3 | 0 | 0 | 1 | Unclassified |
| OTU436 | 4 | 0 | 0 | 0 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_; |
| | | | | | s_ |
| OTU437 | 2 | 3 | 1 | 1 | k_Bacteria; |
| | | | | | p_Planctomycetes; |
| | | | | | c_Planctomycetia; |
| | | | | | o_Pirellulales; |
| | | | | | f_Pirellulaceae; |
| | | | | | g_; |
| | | | | | s_ |
| OTU438 | 4 | 1 | 2 | 1 | k_Bacteria; |
| | | | | | p_Proteobacteria |
| OTU439 | 9 | 1 | 1 | 1 | k_Archaea; |
| | | | | | p_Euryarchaeota; |
| | | | | | c_Thermoplasmata; |
| | | | | | o_E2; |
| | | | | | f_Marine |
| OTU440 | 6 | 1 | 0 | 0 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU441 | 3 | 0 | 0 | 0 | k_Bacteria; |
| | | | | | p_Proteobacteria |
| OTU442 | 3 | 0 | 0 | 0 | Unclassified |
| OTU443 | 33 | 16 | 10 | 9 | k_Bacteria; |
| | | | | | p_Proteobacteria; |
| | | | | | c_Alphaproteobacteria; |
| | | | | | o_Rhodobacterales; |
| | | | | | f_Rhodobacteraceae; |
| | | | | | g_Pseudoruegeria; |
| | | | | | s_ |
| OTU444 | 4 | 0 | 0 | 0 | Unclassified |
| OTU445 | 5 | 3 | 1 | 3 | k_Bacteria; |
| | | | | | p_Bacteroidetes; |
| | | | | | c_[Saprospirae]; |
| | | | | | o_[Saprospirales]; |
| | | | | | f_Saprospiraceae |
| OTU446 | 3 | 0 | 0 | 0 | k_Bacteria; |
| | | | | | p_Planctomycetes; |
| | | | | | c_OM190; |
| | | | | | o_agg27; |
| | | | | | f_; |
| | | | | | g_; |
| | | | | | s_ |
| OTU447 | 2 | 0 | 0 | 1 | Unclassified |
| OTU448 | 7 | 0 | 0 | 0 | k_Bacteria |
| OTU449 | 2 | 2 | 0 | 2 | Unclassified |
| OTU450 | 4 | 0 | 1 | 1 | k_Bacteria; |
| | | | | | p_Actinobacteria; |
| | | | | | c_Acidimicrobiia; |
| | | | | | o_Acidimicrobiales |
| OTU451 | 12 | 5 | 5 | 5 | k_Bacteria; |
| | | | | | p_Chloroflexi; |
| | | | | | c_Anaerolineae |
| OTU452 | 13 | 5 | 2 | 0 | k_Bacteria; |
| | | | | | p_Proteobacteria; |
| | | | | | c_Alphaproteobacteria |
| OTU453 | 20 | 9 | 2 | 1 | k_Archaea; |
| | | | | | p_Euryarchaeota; |
| | | | | | c_Thermoplasmata; |
| | | | | | o_E2; |
| | | | | | f_Marine |
| OTU454 | 4 | 3 | 2 | 0 | k_Bacteria |
| OTU455 | 2 | 1 | 0 | 1 | k_Bacteria |
| OTU456 | 4 | 5 | 1 | 0 | k_Bacteria; |
| | | | | | p_Proteobacteria; |
| | | | | | c_Deltaproteobacteria; |
| | | | | | o_Myxococcales; |
| | | | | | f_OM27; |
| | | | | | g_; |
| | | | | | s_ |
| OTU457 | 5 | 0 | 0 | 2 | k_Bacteria; |
| | | | | | p_Proteobacteria; |
| | | | | | c_Alphaproteobacteria; |
| | | | | | o_Rickettsiales; |
| | | | | | f_mitochondria |
| OTU458 | 4 | 0 | 1 | 3 | k_Bacteria; |
| | | | | | p_Proteobacteria; |
| | | | | | c_Gammaproteobacteria; |
| | | | | | o_Xanthomonadales; |
| | | | | | f_Xanthomonadaceae; |
| | | | | | g_Arenimonas; |
| | | | | | s_oryziterrae |
| OTU459 | 3 | 0 | 0 | 0 | k_Bacteria; |
| | | | | | p_Proteobacteria; |
| | | | | | c_Epsilonproteobacteria; |
| | | | | | o_Campylobacterales; |
| | | | | | f_Helicobacteraceae; |
| | | | | | g_; |
| | | | | | s_ |
| OTU460 | 0 | 7 | 3 | 1 | k_Bacteria |
| OTU461 | 0 | 3 | 2 | 0 | k_Bacteria; |
| | | | | | p_Proteobacteria; |
| | | | | | c_Gammaproteobacteria; |
| | | | | | o_Pseudomonadales; |
| | | | | | f_Moraxellaceae; |
| | | | | | g_Acinetobacter |
| OTU462 | 991 | 953 | 1,104 | 648 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU463 | 0 | 2 | 0 | 0 | k_Bacteria |
| OTU464 | 0 | 2 | 1 | 0 | k_Bacteria |
| OTU465 | 0 | 2 | 0 | 0 | k_Bacteria; |
| | | | | | p_Proteobacteria; |
| | | | | | c_Alphaproteobacteria; |
| | | | | | o_Rhodobacterales |
| OTU466 | 0 | 3 | 0 | 0 | k_Bacteria |
| OTU467 | 0 | 4 | 0 | 0 | Unclassified |
| OTU468 | 0 | 2 | 1 | 0 | Unclassified |
| OTU469 | 1 | 4 | 0 | 0 | Unclassified |
| OTU470 | 4 | 6 | 4 | 2 | k_Bacteria; |
| | | | | | p_Cyanobacteria; |
| | | | | | c_Chloroplast; |
| | | | | | o_Stramenopiles; |
| | | | | | f_; |
| | | | | | g_; |
| | | | | | s_ |
| OTU471 | 2 | 3 | 2 | 1 | k_Bacteria |
| OTU472 | 0 | 2 | 0 | 0 | Unclassified |
| OTU473 | 0 | 1 | 0 | 0 | Unclassified |
| OTU474 | 0 | 3 | 0 | 0 | k_Bacteria |
| OTU475 | 0 | 2 | 0 | 0 | k_Bacteria; |
| | | | | | p_Proteobacteria; |
| | | | | | c_Alphaproteobacteria |
| OTU476 | 0 | 3 | 0 | 0 | k_Bacteria |
| OTU477 | 0 | 2 | 0 | 0 | Unclassified |
| OTU478 | 0 | 4 | 0 | 1 | k_Bacteria; |
| | | | | | p_Bacteroidetes; |
| | | | | | c_Cytophagia; |
| | | | | | o_Cytophagales; |
| | | | | | f_Cytophagaceae; |
| | | | | | g_Hymenobacter; |
| | | | | | s_ |
| OTU479 | 2 | 4 | 3 | 2 | k_Bacteria |
| OTU480 | 0 | 4 | 0 | 0 | k_Bacteria |
| OTU481 | 0 | 2 | 0 | 0 | k_Bacteria |
| OTU482 | 0 | 3 | 0 | 0 | Unclassified |
| OTU483 | 4 | 3 | 0 | 0 | k_Bacteria; |
| | | | | | p_Bacteroidetes |
| OTU484 | 1 | 8 | 0 | 0 | Unclassified |
| OTU485 | 8 | 3 | 3 | 5 | k_Bacteria; |
| | | | | | p_Bacteroidetes; |
| | | | | | c_Flavobacteriia; |
| | | | | | o_Flavobacteriales; |
| | | | | | f_Flavobacteriaceae |
| OTU486 | 0 | 6 | 0 | 3 | k_Bacteria; |
| | | | | | p_Proteobacteria; |
| | | | | | c_Alphaproteobacteria; |
| | | | | | o_Sphingomonadales; |
| | | | | | f_Sphingomonadaceae; |
| | | | | | g_Sphingomonas |
| OTU487 | 0 | 3 | 0 | 0 | k_Bacteria; |
| | | | | | p_Bacteroidetes; |
| | | | | | c_[Saprospirae]; |
| | | | | | o_[Saprospirales]; |
| | | | | | f_Saprospiraceae; |
| | | | | | g_; |
| | | | | | s_ |
| OTU488 | 1 | 3 | 0 | 0 | k_Bacteria; |
| | | | | | p_Bacteroidetes; |
| | | | | | c_[Saprospirae]; |
| | | | | | o_[Saprospirales]; |
| | | | | | f_; |
| | | | | | g_; |
| | | | | | s_ |
| OTU489 | 0 | 2 | 2 | 0 | k_Bacteria; |
| | | | | | p_Bacteroidetes; |
| | | | | | c_Flavobacteriia; |
| | | | | | o_Flavobacteriales; |
| | | | | | f_[Weeksellaceae]; |
| | | | | | g_Chryseobacterium; |
| | | | | | s_ |
| OTU490 | 0 | 2 | 0 | 0 | k_Bacteria |
| OTU491 | 0 | 3 | 0 | 0 | k_Bacteria; |
| | | | | | p_Proteobacteria; |
| | | | | | c_Betaproteobacteria; |
| | | | | | o_Burkholderiales; |
| | | | | | f_Comamonadaceae |
| OTU492 | 1 | 3 | 0 | 0 | k_Bacteria |
| OTU493 | 0 | 2 | 0 | 1 | k_Bacteria; |
| | | | | | p_Bacteroidetes; |
| | | | | | c_Cytophagia; |
| | | | | | o_Cytophagales; |
| | | | | | f_Cytophagaceae; |
| | | | | | g_Hymenobacter; |
| | | | | | s_ |
| OTU494 | 8 | 5 | 3 | 1 | k_Bacteria; |
| | | | | | p_Proteobacteria; |
| | | | | | c_Gammaproteobacteria; |
| | | | | | o_Pseudomonadales; |
| | | | | | f_Moraxellaceae |
| OTU495 | 0 | 4 | 0 | 0 | k_Bacteria |
| OTU496 | 5 | 6 | 3 | 2 | k_Bacteria; |
| | | | | | p_Proteobacteria; |
| | | | | | c_Gammaproteobacteria; |
| | | | | | o_Alteromonadales; |
| | | | | | f_Alteromonadaceae; |
| | | | | | g_HTCC2207; |
| | | | | | s_ |
| OTU497 | 0 | 3 | 0 | 0 | k_Bacteria |
| OTU498 | 316 | 298 | 164 | 237 | k_Bacteria; |
| | | | | | p_Proteobacteria; |
| | | | | | c_Gammaproteobacteria; |
| | | | | | o_Enterobacteriales; |
| | | | | | f_Enterobacteriaceae |
| OTU499 | 1 | 2 | 4 | 1 | k_Bacteria; |
| | | | | | p_Proteobacteria; |
| | | | | | c_Gammaproteobacteria; |
| | | | | | o_Pseudomonadales; |
| | | | | | f_Moraxellaceae; |
| | | | | | g_Acinetobacter |
| OTU500 | 0 | 2 | 0 | 0 | k_Bacteria |
| OTU501 | 0 | 4 | 1 | 2 | k_Bacteria; |
| | | | | | p_Proteobacteria; |
| | | | | | c_Gammaproteobacteria; |
| | | | | | o_; |
| | | | | | f_; |
| | | | | | g_; |
| | | | | | s_ |
| OTU502 | 6 | 7 | 0 | 0 | k_Bacteria; |
| | | | | | p_Proteobacteria; |
| | | | | | c_Alphaproteobacteria; |
| | | | | | o_Kiloniellales; |
| | | | | | f_; |
| | | | | | g_; |
| | | | | | s_ |
| OTU503 | 2 | 2 | 0 | 0 | k_Bacteria; |
| | | | | | p_Proteobacteria; |
| | | | | | c_Gammaproteobacteria |
| OTU504 | 0 | 0 | 4 | 0 | Unclassified |
| OTU505 | 0 | 0 | 4 | 0 | k_Bacteria |
| OTU506 | 0 | 0 | 3 | 0 | k_Bacteria |
| OTU507 | 1 | 0 | 3 | 0 | Unclassified |
| OTU508 | 1 | 0 | 3 | 0 | k_Bacteria; |
| | | | | | p_Chloroflexi; |
| | | | | | c_Anaerolineae; |
| | | | | | o_Caldilineales; |
| | | | | | f_Caldilineaceae |
| OTU509 | 4 | 2 | 5 | 1 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU510 | 0 | 0 | 3 | 0 | Unclassified |
| OTU511 | 0 | 0 | 3 | 0 | k_Bacteria |
| OTU512 | 0 | 0 | 2 | 8 | k_Bacteria |
| OTU513 | 0 | 0 | 3 | 0 | k_Bacteria; |
| | | | | | p_Proteobacteria; |
| | | | | | c_Betaproteobacteria; |
| | | | | | o_Burkholderiales; |
| | | | | | f_Alcaligenaceae; |
| | | | | | g_Sutterella; |
| | | | | | s_ |
| OTU514 | 4 | 12 | 13 | 2 | k_Bacteria; |
| | | | | | p_Firmicutes; |
| | | | | | c_Bacilli; |
| | | | | | o_Lactobacillales; |
| | | | | | f_Leuconostocaceae |
| OTU515 | 3 | 7 | 10 | 2 | k_Bacteria; |
| | | | | | p_Proteobacteria; |
| | | | | | c_Gammaproteobacteria; |
| | | | | | o_Pseudomonadales; |
| | | | | | f_Moraxellaceae; |
| | | | | | g_Acinetobacter; |
| | | | | | s_guillouiae |
| OTU516 | 0 | 0 | 3 | 0 | k_Bacteria |
| OTU517 | 0 | 0 | 3 | 0 | k_Bacteria; |
| | | | | | p_Planctomycetes; |
| | | | | | c_BD7-11; |
| | | | | | o_; |
| | | | | | f_; |
| | | | | | g_; |
| | | | | | s_ |
| OTU518 | 0 | 0 | 9 | 3 | k_Bacteria |
| OTU519 | 0 | 0 | 3 | 0 | k_Bacteria |
| OTU520 | 0 | 0 | 2 | 1 | k_Bacteria |
| OTU521 | 0 | 1 | 3 | 0 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_Capnocytophaga |
| OTU522 | 0 | 0 | 3 | 2 | k_Bacteria; |
| | | | | | p_Proteobacteria; |
| | | | | | c_Alphaproteobacteria; |
| | | | | | o_Rickettsiales; |
| | | | | | f_mitochondria |
| OTU523 | 1 | 0 | 83 | 43 | k_Bacteria; |
| | | | | | p_Firmicutes; |
| | | | | | c_Clostridia; |
| | | | | | o_Clostridiales; |
| | | | | | f_Lachnospiraceae |
| OTU524 | 0 | 0 | 3 | 0 | k_Bacteria |
| OTU525 | 0 | 0 | 2 | 0 | k_Bacteria |
| OTU526 | 0 | 0 | 3 | 0 | k_Bacteria |
| OTU527 | 0 | 0 | 2 | 1 | k_Bacteria |
| OTU528 | 0 | 0 | 3 | 2 | k_Bacteria |
| OTU529 | 0 | 0 | 4 | 0 | Unclassified |
| OTU530 | 0 | 0 | 2 | 1 | k_Bacteria |
| OTU531 | 0 | 0 | 1 | 1 | k_Bacteria |
| OTU532 | 0 | 0 | 3 | 2 | Unclassified |
| OTU533 | 0 | 0 | 3 | 1 | k_Bacteria |
| OTU534 | 0 | 0 | 0 | 5 | k_Bacteria |
| OTU535 | 0 | 0 | 0 | 5 | k_Bacteria; |
| | | | | | p_Proteobacteria; |
| | | | | | c_Alphaproteobacteria; |
| | | | | | o_Rickettsiales; |
| | | | | | f_mitochondria; |
| | | | | | g_Dictyostelium |
| OTU536 | 0 | 0 | 0 | 4 | k_Bacteria; |
| | | | | | p_Proteobacteria |
| OTU537 | 0 | 0 | 1 | 6 | Unclassified |
| OTU538 | 0 | 0 | 1 | 3 | Unclassified |
| OTU539 | 0 | 0 | 15 | 11 | k_Bacteria; |
| | | | | | p_Firmicutes; |
| | | | | | c_Clostridia; |
| | | | | | o_Clostridiales; |
| | | | | | f_Peptostreptococcaceae; |
| | | | | | g_Clostridium; |
| | | | | | s_venationis |
| OTU540 | 0 | 0 | 0 | 3 | k_Bacteria |
| OTU541 | 0 | 0 | 0 | 8 | k_Bacteria |
| OTU542 | 0 | 0 | 1 | 3 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_Winogradskyella; |
| | | | | | s_ |
| OTU543 | 7 | 11 | 14 | 113 | k_Bacteria; |
| | | | | | p_Firmicutes; |
| | | | | | c_Clostridia; |
| | | | | | o_Clostridiales; |
| | | | | | f_Veillonellaceae; |
| | | | | | g_Veillonella |
| OTU544 | 0 | 0 | 0 | 4 | k_Bacteria |
| OTU545 | 0 | 0 | 0 | 9 | k_Bacteria |
| OTU546 | 0 | 0 | 0 | 3 | k_Bacteria |
| OTU547 | 0 | 0 | 0 | 3 | k_Bacteria; |
| | | | | | p_Proteobacteria; |
| | | | | | c_Alphaproteobacteria; |
| | | | | | o_Rickettsiales; |
| | | | | | f_mitochondria |
| OTU548 | 0 | 0 | 0 | 8 | k_Bacteria |
| OTU549 | 1 | 0 | 0 | 8 | k_Bacteria; |
| | | | | | p_Firmicutes; |
| | | | | | c_Clostridia; |
| | | | | | o_Clostridiales; |
| | | | | | f_Lachnospiraceae |
| OTU550 | 0 | 0 | 0 | 3 | k_Bacteria |
| OTU551 | 0 | 0 | 0 | 3 | k_Bacteria |
| OTU552 | 0 | 0 | 0 | 3 | k_Bacteria |
| OTU553 | 0 | 1 | 1 | 4 | k_Bacteria; |
| | | | | | p_Bacteroidetes; |
| | | | | | c_[Saprospirae]; |
| | | | | | o_[Saprospirales]; |
| | | | | | f_Saprospiraceae |
| OTU554 | 0 | 0 | 0 | 3 | Unclassified |
| OTU555 | 0 | 0 | 0 | 3 | k_Bacteria |
| OTU556 | 0 | 1 | 0 | 2 | k_Bacteria; |
| | | | | | p_Proteobacteria; |
| | | | | | c_Alphaproteobacteria; |
| | | | | | o_Ellin329; |
| | | | | | f_; |
| | | | | | g_; |
| | | | | | s_ |
| OTU557 | 0 | 0 | 1 | 8 | Unclassified |
| OTU558 | 0 | 5 | 0 | 35 | k_Bacteria; |
| | | | | | p_Bacteroidetes; |
| | | | | | c_Bacteroidia; |
| | | | | | o_Bacteroidales; |
| | | | | | f_Prevotellaceae; |
| | | | | | g_Prevotella; |
| | | | | | s_ |
| OTU559 | 0 | 0 | 2 | 10 | k_Bacteria; |
| | | | | | p_Firmicutes; |
| | | | | | c_Clostridia; |
| | | | | | o_Clostridiales; |
| | | | | | f_[Mogibacteriaceae]; |
| | | | | | g_; |
| | | | | | s_ |
| OTU560 | 0 | 0 | 0 | 3 | k_Bacteria |
| OTU561 | 0 | 0 | 1 | 4 | k_Bacteria; |
| | | | | | p_Proteobacteria; |
| | | | | | c_Alphaproteobacteria; |
| | | | | | o_Rhodospirillales; |
| | | | | | f_; |
| | | | | | g_; |
| | | | | | s_ |
| OTU562 | 0 | 0 | 0 | 3 | k_Bacteria |
| OTU563 | 3 | 1 | 0 | 3 | k_Bacteria; |
| | | | | | p_Chloroflexi; |
| | | | | | c_Anaerolineae |
| OTU564 | 0 | 0 | 0 | 4 | k_Bacteria |
| OTU565 | 1 | 0 | 0 | 2 | k_Bacteria; |
| | | | | | p_Proteobacteria; |
| | | | | | c_Alphaproteobacteria |
| OTU566 | 0 | 0 | 0 | 3 | k_Bacteria |
| OTU567 | 0 | 1 | 1 | 4 | k_Bacteria |
| OTU568 | 0 | 0 | 0 | 3 | k_Bacteria |
| OTU569 | 0 | 0 | 3 | 9 | k_Bacteria |
| OTU570 | 0 | 0 | 0 | 6 | k_Bacteria |
| OTU571 | 0 | 0 | 0 | 3 | k_Bacteria |
| OTU572 | 0 | 0 | 0 | 5 | k_Bacteria |
| OTU573 | 0 | 0 | 0 | 2 | k_Bacteria |
| OTU574 | 0 | 0 | 0 | 4 | k_Bacteria; |
| | | | | | p_Actinobacteria; |
| | | | | | c_Coriobacteriia; |
| | | | | | o_Coriobacteriales; |
| | | | | | f_Coriobacteriaceae; |
| | | | | | g_Collinsella; |
| | | | | | s_aerofaciens |
| OTU575 | 0 | 0 | 0 | 3 | k_Bacteria |
| OTU576 | 0 | 0 | 0 | 4 | k_Bacteria |
| OTU577 | 0 | 0 | 0 | 3 | k_Bacteria; |
| | | | | | p_Proteobacteria; |
| | | | | | c_Alphaproteobacteria; |
| | | | | | o_Rickettsiales; |
| | | | | | f_mitochondria |
| OTU578 | 0 | 0 | 0 | 6 | k_Bacteria |
| OTU579 | 0 | 0 | 0 | 3 | k_Bacteria; |
| | | | | | p_Proteobacteria; |
| | | | | | c_Gammaproteobacteria; |
| | | | | | o_Xanthomonadales; |
| | | | | | f_Xanthomonadaceae; |
| | | | | | g_Thermomonas; |
| | | | | | s_ |
| OTU580 | 0 | 0 | 0 | 1 | k_Bacteria |
| OTU581 | 0 | 0 | 0 | 6 | k_Bacteria |
| OTU582 | 2 | 1 | 0 | 4 | k_Bacteria; |
| | | | | | p_Bacteroidetes; |
| | | | | | c_Flavobacteriia; |
| | | | | | o_Flavobacteriales; |
| | | | | | f_Flavobacteriaceae; |
| | | | | | g_; |
| | | | | | s_ |
| OTU583 | 0 | 0 | 0 | 4 | k_Bacteria; |
| | | | | | p_Proteobacteria; |
| | | | | | c_Alphaproteobacteria |
| OTU584 | 0 | 0 | 0 | 2 | k_Bacteria |
| OTU585 | 0 | 0 | 0 | 3 | k_Bacteria |
| OTU586 | 0 | 0 | 0 | 3 | k_Bacteria |
| OTU587 | 0 | 0 | 0 | 4 | k_Bacteria |
| OTU588 | 2 | 0 | 0 | 2 | k_Bacteria; |
| | | | | | p_Proteobacteria; |
| | | | | | c_Gammaproteobacteria |
| OTU589 | 0 | 0 | 0 | 3 | k_Bacteria |
| OTU590 | 3 | 0 | 2 | 3 | Unclassified |
| OTU591 | 1 | 0 | 0 | 3 | k_Bacteria; |
| | | | | | p_Proteobacteria; |
| | | | | | c_Alphaproteobacteria; |
| | | | | | o_Rhizobiales; |
| | | | | | f_; |
| | | | | | g_; |
| | | | | | s_ |
| OTU592 | 0 | 0 | 0 | 2 | k_Bacteria; |
| | | | | | p_Proteobacteria |
| OTU593 | 0 | 0 | 0 | 2 | Unclassified |
| OTU594 | 0 | 2 | 0 | 3 | k_Bacteria; |
| | | | | | p_Proteobacteria; |
| | | | | | c_Alphaproteobacteria; |
| | | | | | o_Rhizobiales; |
| | | | | | f_Hyphomicrobiaceae; |
| | | | | | g_Devosia; |
| | | | | | s_ |
| OTU595 | 0 | 0 | 0 | 13 | k_Bacteria; |
| | | | | | p_Actinobacteria; |
| | | | | | c_Coriobacteriia; |
| | | | | | o_Coriobacteriales; |
| | | | | | f_Coriobacteriaceae; |
| | | | | | g_Atopobium; |
| | | | | | s_ |
| OTU596 | 0 | 0 | 0 | 2 | k_Bacteria; |
| | | | | | p_Proteobacteria; |
| | | | | | c_Alphaproteobacteria; |
| | | | | | o_Rhodobacterales; |
| | | | | | f_Rhodobacteraceae |
| OTU597 | 10 | 1 | 0 | 5 | k_Bacteria; |
| | | | | | p_Bacteroidetes; |
| | | | | | c_[Rhodothermi]; |
| | | | | | o_[Rhodothermales]; |
| | | | | | f_[Balneolaceae]; |
| | | | | | g_Balneola; |
| | | | | | s_ |
| OTU598 | 0 | 0 | 0 | 5 | k_Bacteria; |
| | | | | | p_Actinobacteria; |
| | | | | | c_Coriobacteriia; |
| | | | | | o_Coriobacteriales; |
| | | | | | f_Coriobacteriaceae; |
| | | | | | g_; |
| | | | | | s_ |
| OTU599 | 0 | 0 | 0 | 4 | k_Bacteria; |
| | | | | | p_Firmicutes; |
| | | | | | c_Clostridia; |
| | | | | | o_Clostridiales; |
| | | | | | f_Peptostreptococcaceae; |
| | | | | | g_Clostridium; |
| | | | | | s_venationis |
| OTU600 | 3 | 2 | 9 | 33 | k_Bacteria; |
| | | | | | p_Firmicutes; |
| | | | | | c_Clostridia; |
| | | | | | o_Clostridiales; |
| | | | | | f_Lachnospiraceae |
| OTU601 | 0 | 0 | 0 | 3 | k_Bacteria |

These sequencing results show clear changes in the microbial consortium from the original sample (represented by samples 1 and 2) to the consortium after the treatments with the growth medium and metal exposure described above (sample 4 represents the consortium after the treatment, and sample 3 represents the consortium after partial treatment). For example, large increases were observed in the abundance of OTU45 (bacteria of the Lachnospiraceae family of the Clostridiales order) from 225 in sample 1 to 8,808 in sample 4; OTU51 (Clostridium venationis) from 7 in sample 1 to 5,052 in sample 4; OTU61 (Xanthomonas axonopodis) from 91 in sample 1 to 581 in sample 4; OTU116 (Paenibacillus) from 4 in sample 1 to 2,535 in sample 4; OTU140 (Clostridium hungatei) from 1 in sample 2 to 1,421 in sample 4.

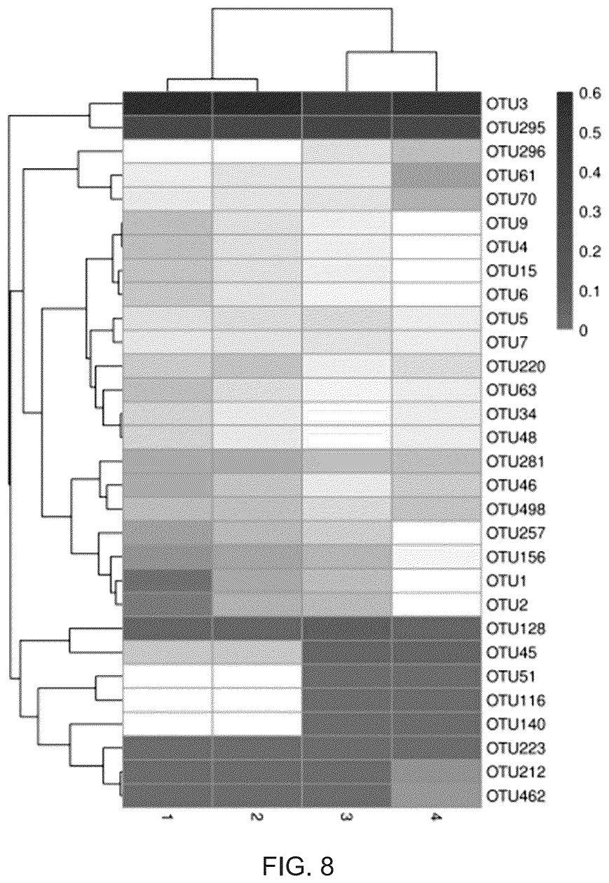

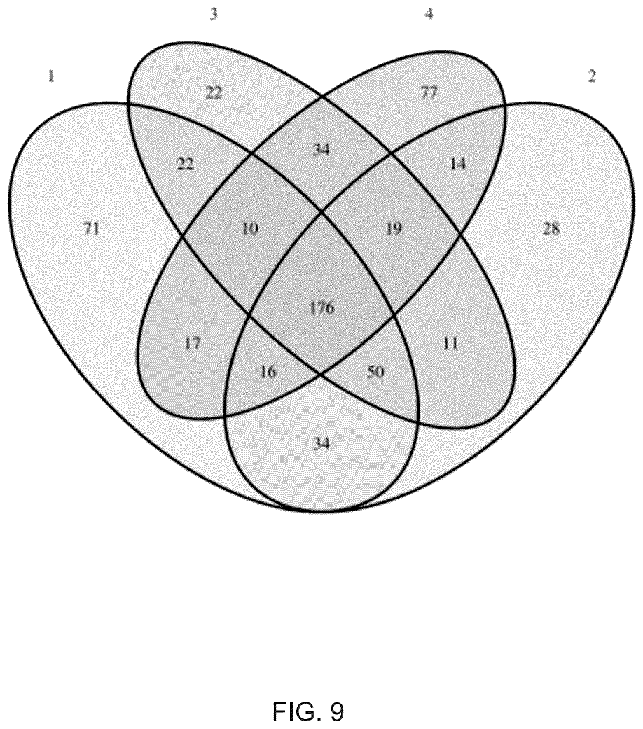

FIG. 8 shows a heat map of the 30 most abundant OTUs. The four columns represent samples 1, 2, 3, and 4. Darker rectangles represent more abundant OTUs and lighter rectangles represent less abundant OTUs. The OTUs are clustered by similarity, with similarity cluster trees shown on the left side of the figure.

FIG. 9 shows a Venn diagram representing the number of OTUs present in samples 1, 2, 3, and 4. The samples are represented by overlapping elipses, and the numbers inside each segment of the overlapping elipses represent the number of OTUs present in the particular combination of samples that overlap in each particular segment.

A representative sequence was selected from each OTU and annotated using the RDP classifier to obtain the community composition of each sample. The RDP classifier Bayesian algorithm was used to classify the OTU representative sequences of 97% similarity level, and the community composition of each sample was analyzed and summarized at all levels. The comparison database was the Greengenes database (available at http://qiime.org/home_static/dataFiles.html). The members of the community in each sample and their relative abundance are shown in Table 3:

TABLE 3

| Taxon | 1 | 2 | 3 | 4 |
|---|---|---|---|---|
| Acinetobacter | 9.00 | 14.00 | 21.00 | 3.00 |
| Arenimonas | 4.00 | 0.00 | 1.00 | 3.00 |
| Atopobium | 0.00 | 0.00 | 0.00 | 13.00 |
| Bacillus | 9.00 | 12.00 | 40.00 | 42.00 |
| Balneola | 10.00 | 1.00 | 0.00 | 5.00 |
| Candidatus | 159.00 | 80.00 | 30.00 | 62.00 |
| Capnocytophaga | 0.00 | 1.00 | 3.00 | 0.00 |
| Chryseobacterium | 2.00 | 7.00 | 5.00 | 0.00 |
| Clostridium | 7 | 7 | 6,084 | 6,531 |
| Collinsella | 0.00 | 0.00 | 0.00 | 4.00 |
| Devosia | 0.00 | 2.00 | 0.00 | 3.00 |
| Dictyostelium | 0.00 | 0.00 | 0.00 | 5.00 |
| Dorea | 0.00 | 0.00 | 143.00 | 293.00 |
| Enterococcus | 43.00 | 9.00 | 3.00 | 6.00 |
| Escherichia | 7.00 | 3.00 | 4.00 | 12.00 |
| Fluviicola | 19.00 | 14.00 | 1.00 | 5.00 |
| Glaciecola | 17.00 | 4.00 | 5.00 | 1.00 |
| Haemophilus | 1.00 | 1.00 | 15.00 | 5.00 |
| HTCC | 8.00 | 5.00 | 2.00 | 3.00 |
| HTCC2207 | 5.00 | 6.00 | 3.00 | 2.00 |
| Hymenobacter | 0.00 | 6.00 | 0.00 | 2.00 |
| Janthinobacterium | 10.00 | 6.00 | 3.00 | 5.00 |
| Lactobacillus | 108.00 | 62.00 | 50.00 | 3.00 |
| Leuconostoc | 16.00 | 8.00 | 22.00 | 6.00 |
| Loktanella | 191.00 | 111.00 | 28.00 | 98.00 |
| MB11C04 | 3.00 | 3.00 | 0.00 | 0.00 |
| Moritella | 4.00 | 0.00 | 2.00 | 1.00 |
| Nitrosopumilus | 127.00 | 66.00 | 31.00 | 17.00 |
| Octadecabacter | 70.00 | 47.00 | 15.00 | 22.00 |
| Oleibacter | 7.00 | 2.00 | 1.00 | 0.00 |
| Paenibacillus | 4.00 | 4.00 | 3,636.00 | 2,535.00 |
| Pantoea | 43.00 | 55.00 | 56.00 | 55.00 |
| Pediococcus | 602.00 | 381.00 | 201.00 | 14.00 |
| Peptostreptococcus | 4.00 | 10.00 | 16.00 | 56.00 |
| Planctomyces | 9.00 | 5.00 | 1.00 | 0.00 |
| Planctomycete | 18.00 | 5.00 | 1.00 | 6.00 |
| Plesiocystis | 1.00 | 0.00 | 0.00 | 4.00 |
| Prevotella | 0.00 | 5.00 | 0.00 | 35.00 |
| Propionigenium | 26.00 | 21.00 | 6.00 | 6.00 |
| Pseudoalteromonas | 16.00 | 10.00 | 1.00 | 4.00 |
| Pseudoruegeria | 33.00 | 16.00 | 10.00 | 9.00 |
| Rhodobacter | 4.00 | 0.00 | 1.00 | 0.00 |
| Rothia | 1.00 | 6.00 | 0.00 | 6.00 |
| Saprospira | 11.00 | 8.00 | 1.00 | 2.00 |
| Sphingomonas | 3.00 | 12.00 | 1.00 | 7.00 |
| Stenotrophomonas | 0.00 | 24.00 | 3.00 | 6.00 |
| Sutterella | 0.00 | 0.00 | 3.00 | 0.00 |
| Tenacibaculum | 37.00 | 19.00 | 5.00 | 25.00 |
| Thermomonas | 0.00 | 0.00 | 0.00 | 3.00 |
| Thiomicrospira | 18.00 | 5.00 | 0.00 | 6.00 |
| Unclassified | 192,843 | 193,403 | 184,017 | 183,979 |
| Veillonella | 7.00 | 11.00 | 14.00 | 113.00 |
| Winogradskyella | 0.00 | 0.00 | 1.00 | 3.00 |
| Xanthomonas | 91.00 | 130.00 | 121.00 | 581.00 |

For each sample, the percentage of species at different taxonomic levels are shown in Table 4:

TABLE 4

| Samples | 1 | 2 | 3 | 4 |
|---|---|---|---|---|
| Class | 26 | 25 | 21 | 25 |
| Family | 53 | 52 | 50 | 50 |
| Genus | 42 | 43 | 42 | 47 |
| Kingdom | 3 | 3 | 3 | 3 |
| Order | 44 | 43 | 37 | 43 |
| Phylum | 13 | 13 | 13 | 12 |
| Species | 12 | 12 | 12 | 13 |

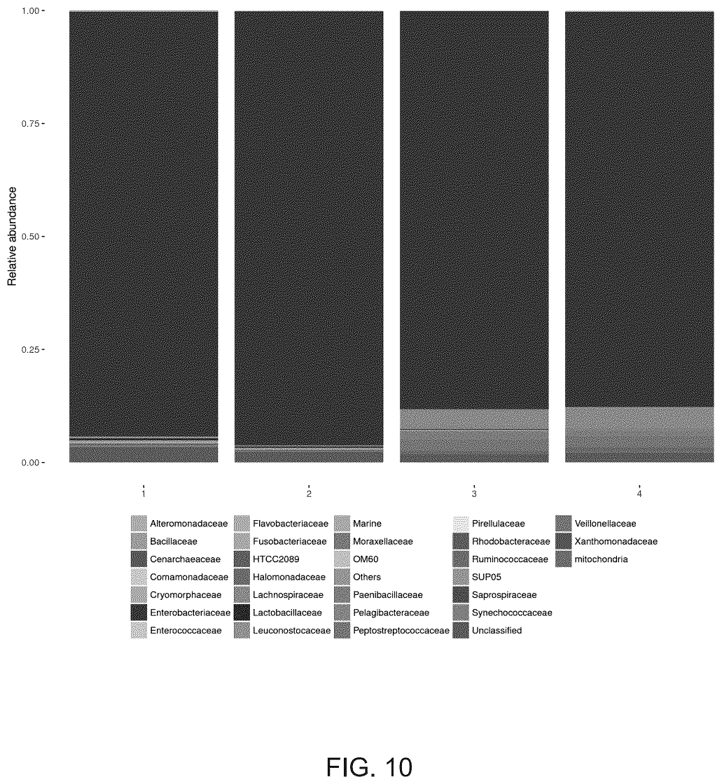

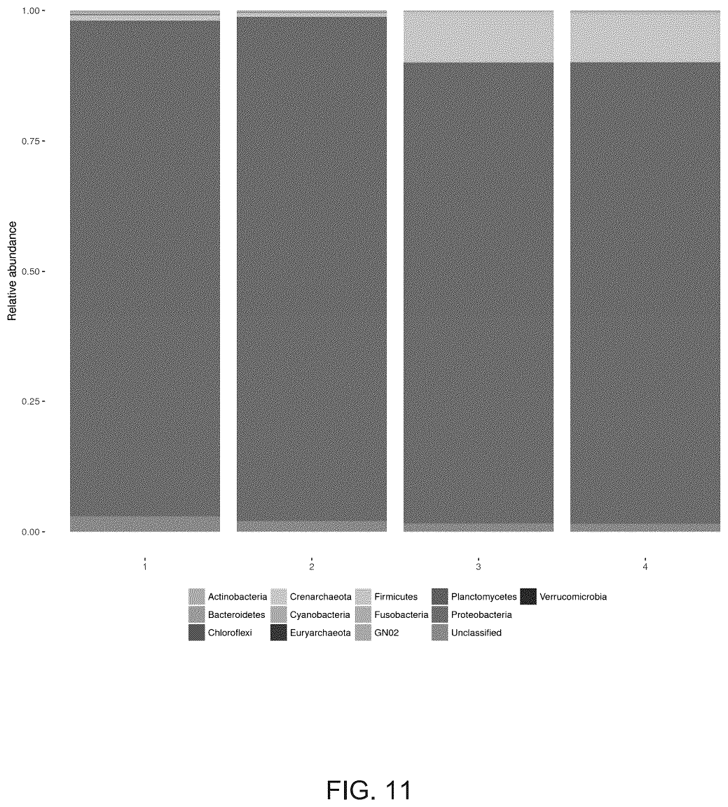

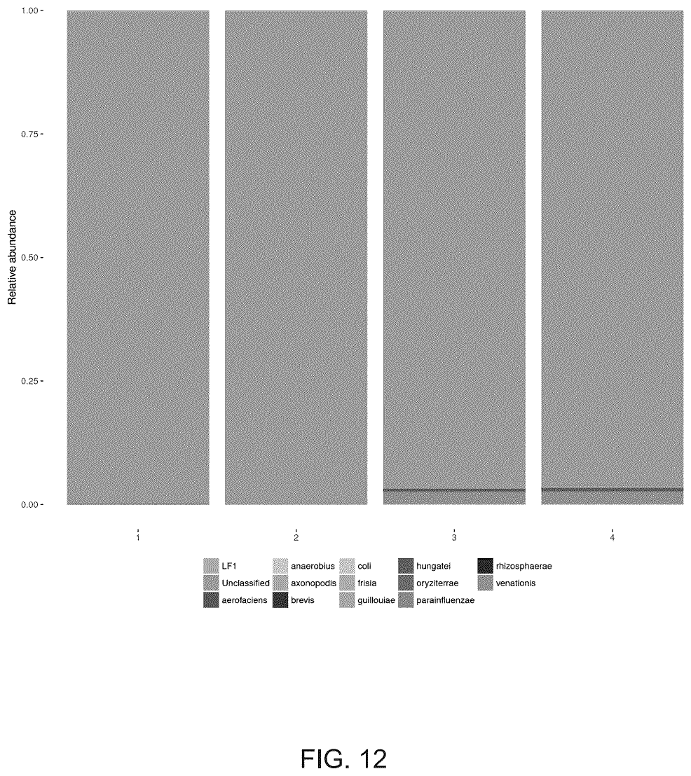

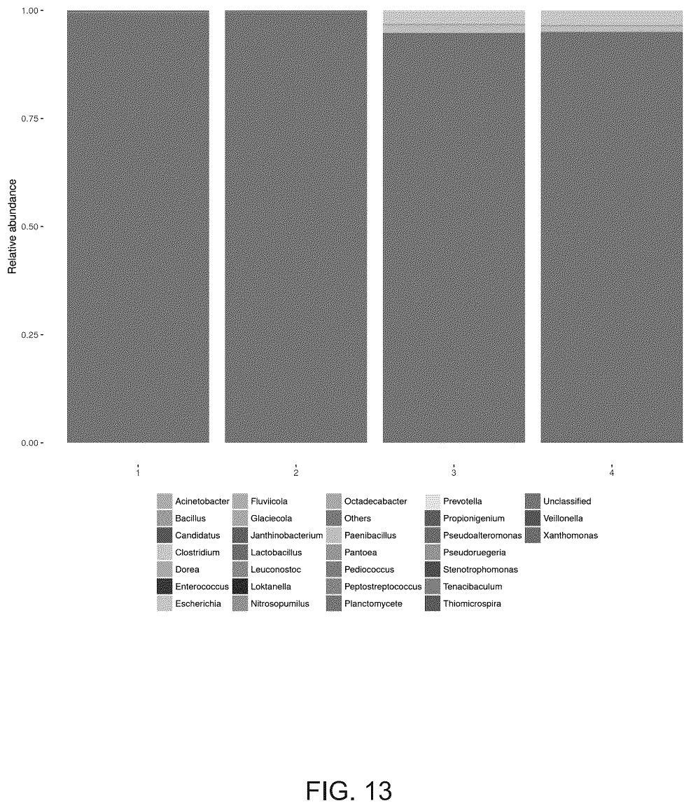

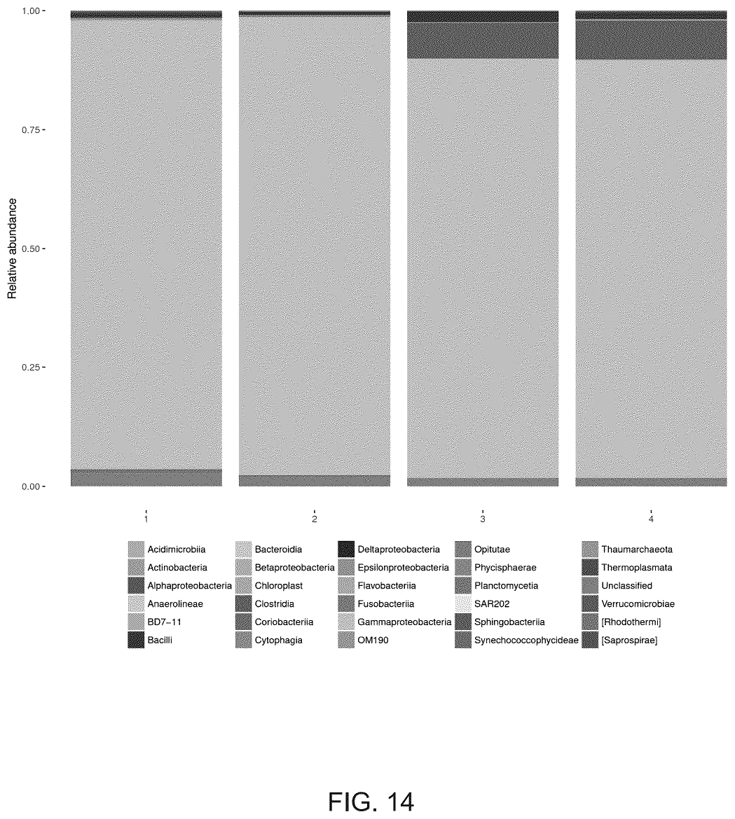

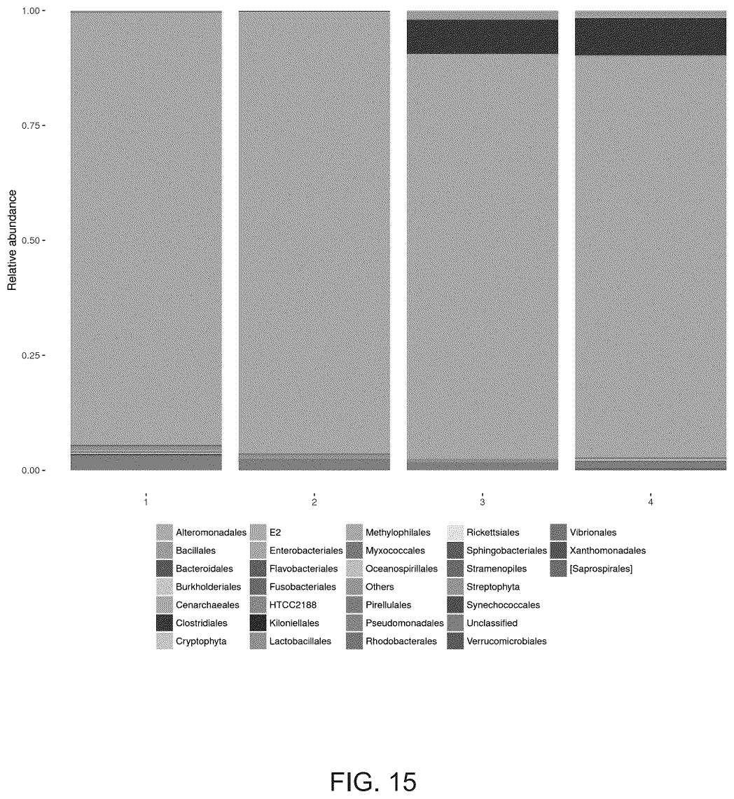

FIGS. 10-15 are bar charts showing the relative abundance of the 30 most abundant classifications in each sample at different taxonomic levels (FIG. 10 shows the phylum level; FIG. 11 shows the class level; FIG. 12 shows the order level; FIG. 13 shows the family level; FIG. 14 shows the genus level; FIG. 15 shows the species level).

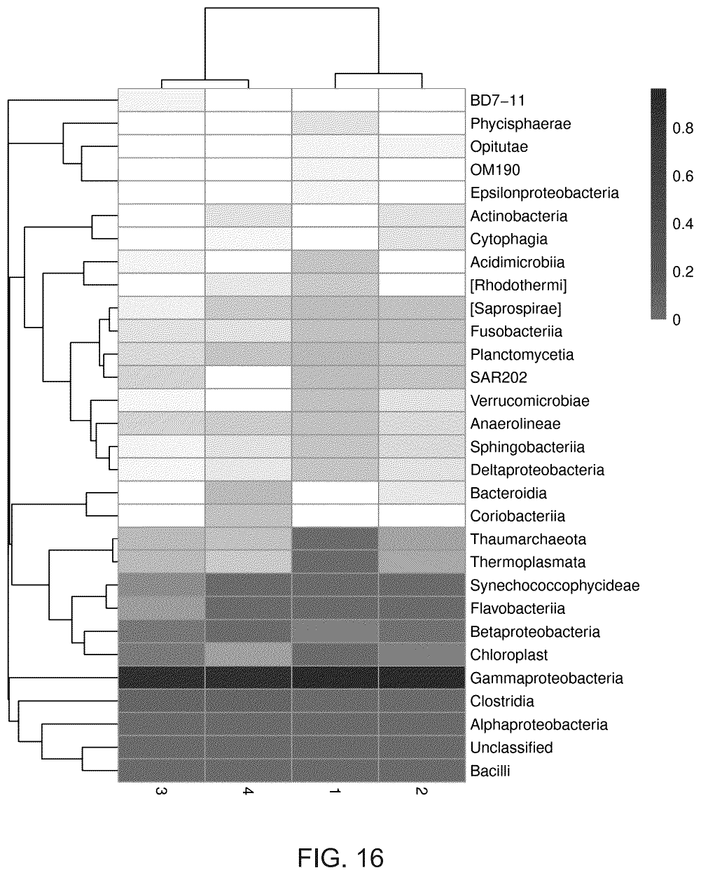

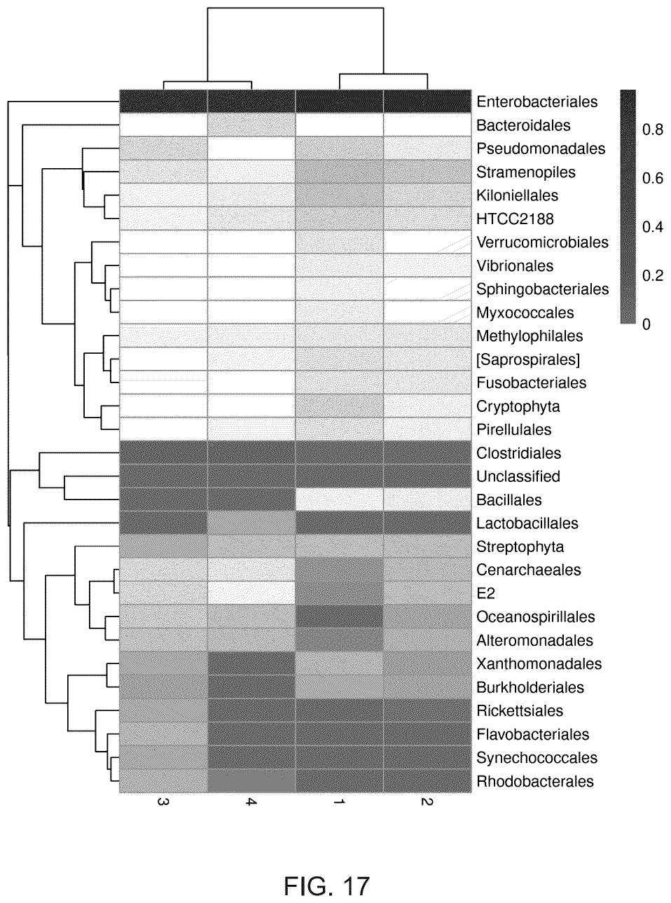

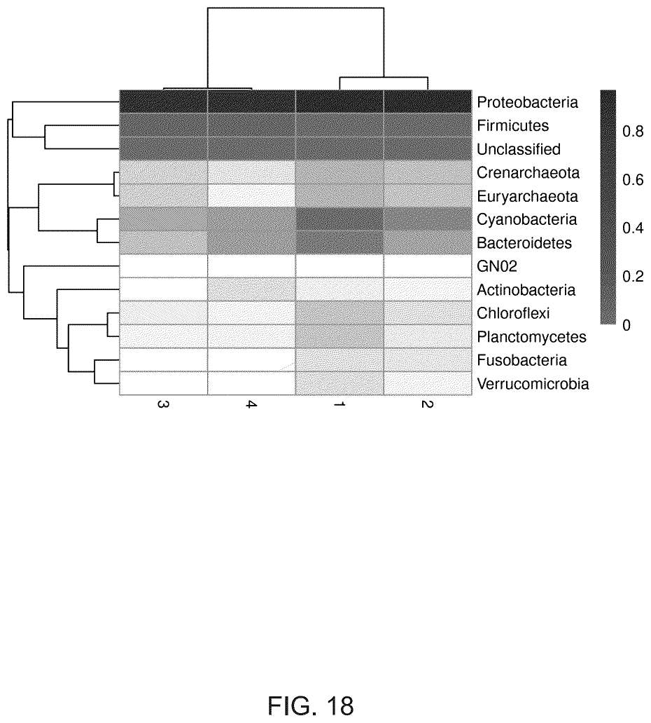

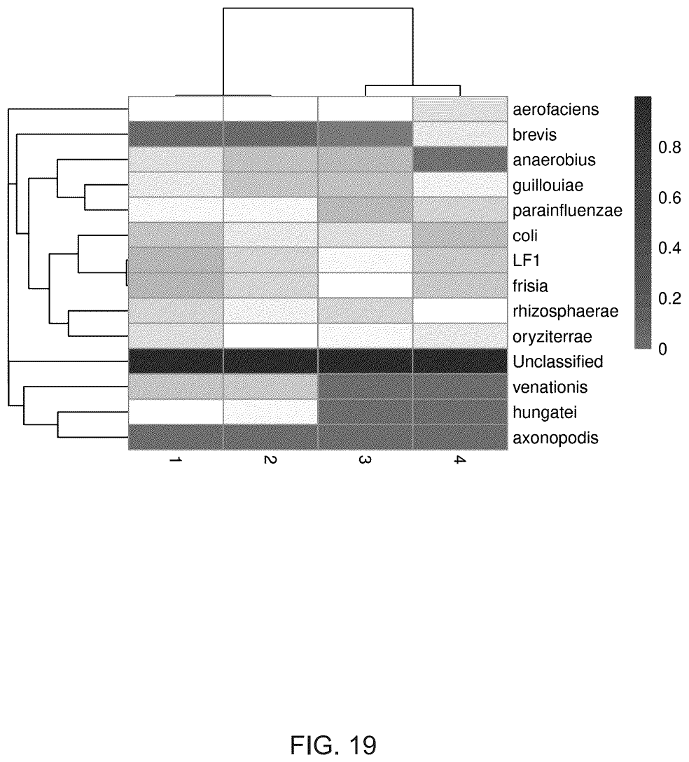

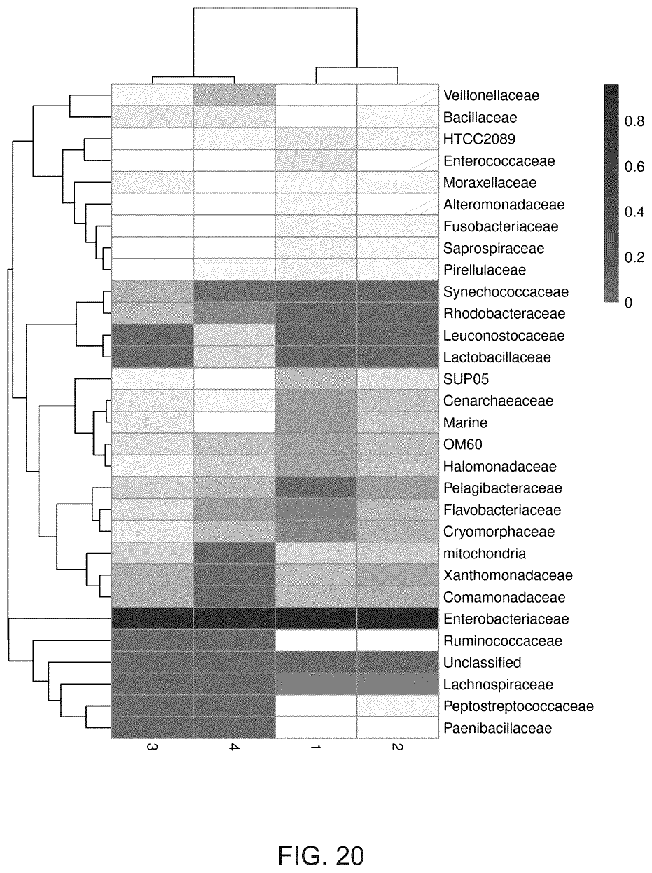

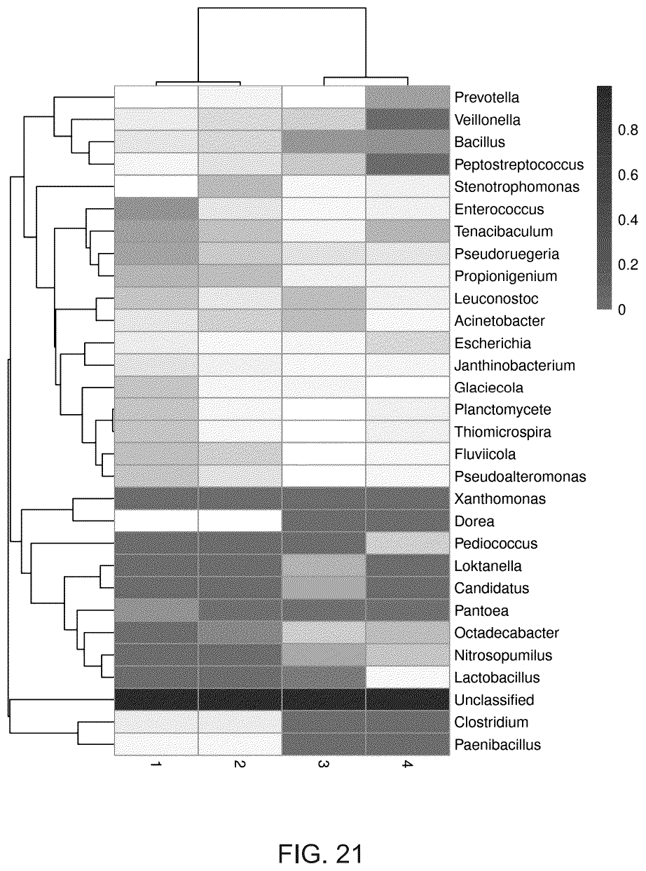

FIGS. 16-21 are heat maps showing the top 30 most abundant classifications in each sample at different taxonomic levels. More abundant classifications are represented by darker rectangles, while less abundant classifications are represented by lighter rectangles. The classifications are clustered by similarity. FIG. 16 shows the phylum level; FIG. 17 shows the class level; FIG. 18 shows the order level; FIG. 19 shows the family level; FIG. 20 shows the genus level; FIG. 21 shows the species level.

Alpha diversity indices were calculated for samples 1, 2, 3, and 4. The alpha diversity indices are statistical indices used to reflect the diversity of the samples. Alpha diversity indices estimate the number of species in the microbial community and the abundance and diversity of species. The indices that were calculated include: ACE, an index to estimate the number of OTUs in a community (see http://www.mothur.org/wiki/Ace); Chao, an index that uses the Chao 1 algorithm to estimate the number of OTUs in a sample (see http://www.mothur.org/wiki/Chao); Shannon, an index for the estimation of microbial diversity (see http://www.mothur.org/wiki/Shannon); Simpson, an index for quantifying biological diversity proposed by Edward Hugh Shannon in 1949 (see http://www.mothur.org/wiki/Simpson); and Goods Coverage, which is an index referring to library coverage of each sample, where a higher value indicates a lower probability that the sample did not cover the sequence (see http://www.mothur.org/wiki/Coverage). The alpha diversity index values for samples 1, 2, 3, and 4 are listed in Table 5:

TABLE 5

| sample | ace | chao1 | shannon | simpson | goods_coverage |
|---|---|---|---|---|---|
| 1 | 402.38 | 403.78 | 2.06 | 0.59 | 1 |
| 2 | 371.91 | 375.02 | 1.84 | 0.57 | 1 |
| 3 | 410.04 | 415.65 | 2.29 | 0.67 | 1 |
| 4 | 392.76 | 394.80 | 2.21 | 0.64 | 1 |

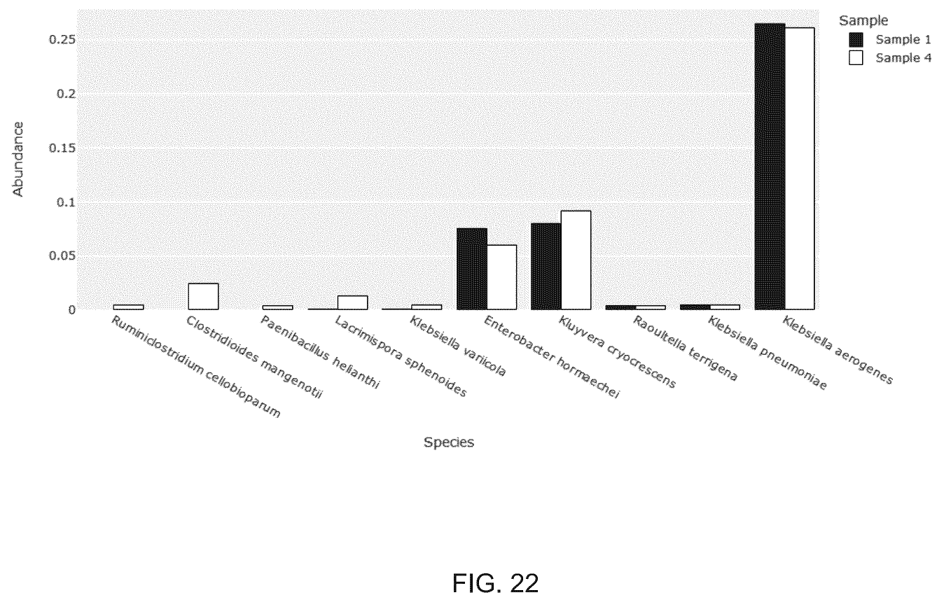

After additional study to identify species and genera of the bacteria, the abundance of the most common identified species and genera was estimated. Significant changes in abundance were observed for many of the species and genera from the starting point of sample 1 to the modified population in sample 4. FIG. 22 is a bar graph showing the estimated abundance of several species in sample 1 compared with the estimated abundance in sample 4. The abundance was calculated by summing the confidence scores (ranging from 0 to 1) for all reads that are classified as a particular species and dividing by the total number of reads in the sample. This figure only shows abundances for species with a total confidence (a.k.a. weighted read total)>=1,000 in either sample 1 or sample 4. These species are ordered left to right from species with the largest magnitude change in abundance between sample 1 and sample 4 to species with the smallest magnitude change.

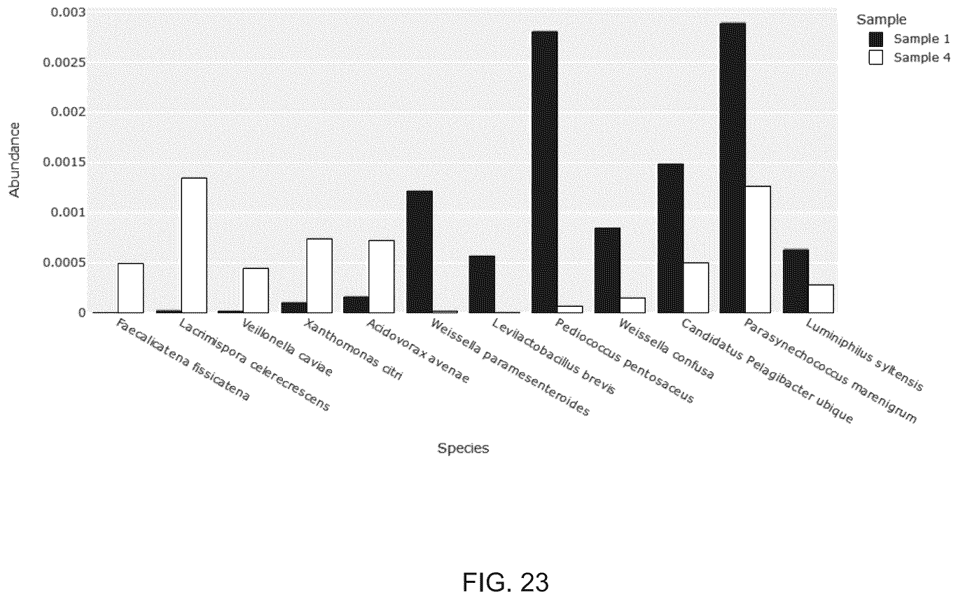

FIG. 23 is a bar graph showing the estimated abundance of several more species in sample 1 compared to sample 4. These species were present at an overall lower abundance compared to the species in FIG. 22, hence the smaller values on the y-axis of the graph. Again, abundance was calculated by summing the confidence scores (ranging from 0 to 1) for all reads that are classified as a particular species and dividing by the total number of reads in the sample. This figure only shows abundances for species with a total confidence (a.k.a. weighted read total)>=100 and <1,000 in either sample 1 or sample 4. These species are ordered left to right from species with the largest magnitude change in abundance between sample 1 and sample 4 to species with the smallest magnitude change.

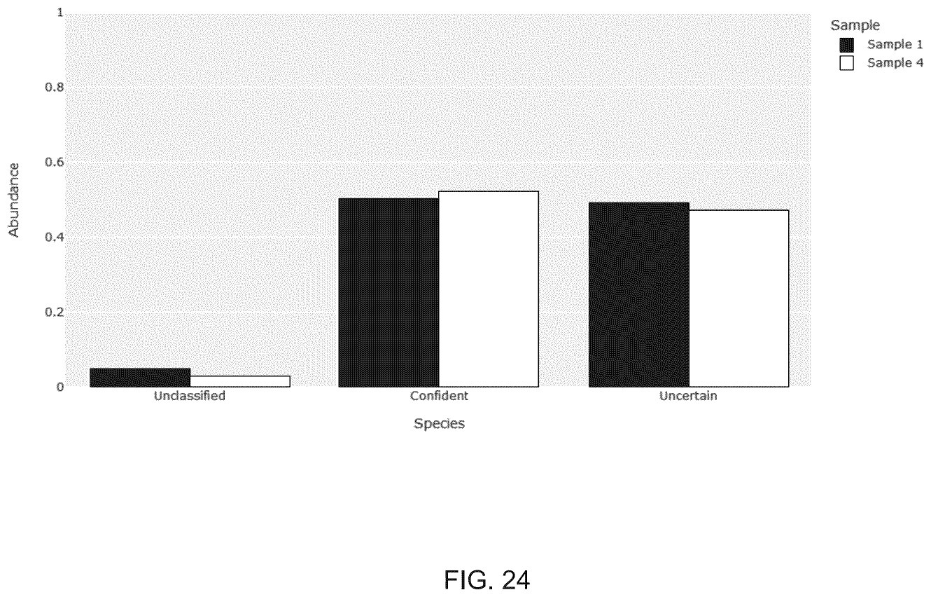

FIG. 24 is a bar graph showing estimated portions of sample 1 and sample 4 that were unclassified as a species, classified with confidence, or classified without confidence (uncertain). The unclassified portion of a sample (left) was calculated by counting the number of unclassified reads and dividing by the total number of reads in the sample. The portion of a sample classified with confidence (middle) was calculated by summing the confidence scores (ranging from 0 to 1) for all classified reads and dividing by the total number of reads in the sample. Finally, the uncertain portion of a sample (right) was calculated by summing the complements of the confidence scores (1—score) for all classified reads and dividing by the total number of reads in the sample.

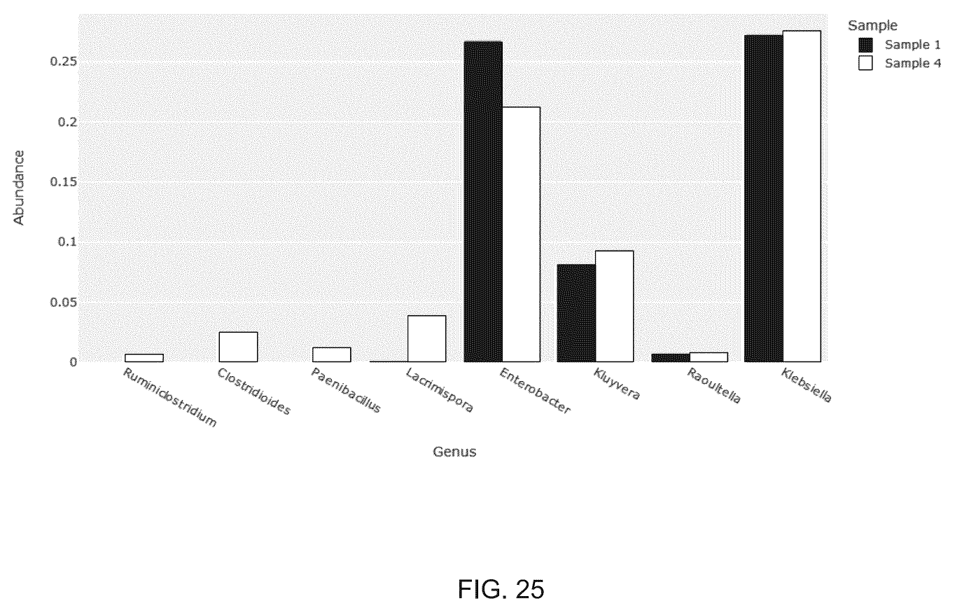

FIG. 25 is a bar graph showing the estimated abundance of genera for several genera (for bacteria that were classified at the genus level) in sample 1 compared with sample 4. Abundance is calculated by summing the confidence scores (ranging from 0 to 1) for all reads that are classified as a particular genus and dividing by the total number of reads in the sample. This figure only shows abundances for genera with a total confidence (a.k.a. weighted read total)>=1,000 in either sample 1 or sample 4. These genera are ordered left to right from genera with the largest magnitude change in abundance between sample 1 and sample 4 to genera with the smallest magnitude change.

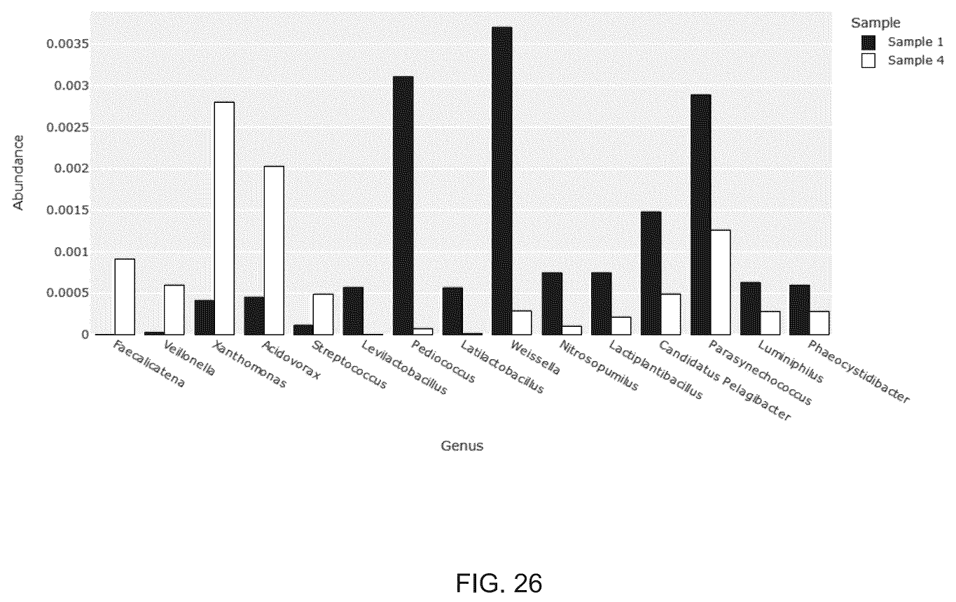

FIG. 26 is another bar graph showing the estimated abundance of several more genera in sample 1 compared with sample 4. Abundance was calculated by summing the confidence scores (ranging from 0 to 1) for all reads that are classified as a particular genus and dividing by the total number of reads in the sample. This figure only shows abundances for genera with a total confidence (a.k.a. weighted read total)>=100 and <1,000 in either sample 1 or sample 4. These genera are ordered left to right from genera with the largest magnitude change in abundance between sample 1 and sample 4 to genera with the smallest magnitude change.

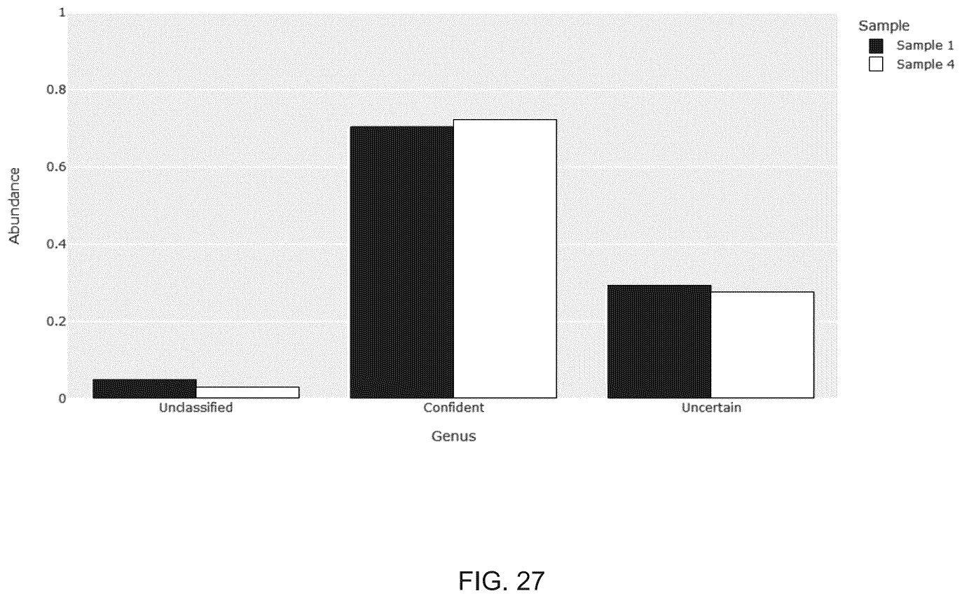

FIG. 27 is a bar graph showing the estimated portions of sample 1 and sample 4 that were unclassified as a genus, classified with confidence, or classified without confidence (uncertain). The unclassified portion of a sample (left) was calculated by counting the number of unclassified reads and dividing by the total number of reads in the sample. The portion of a sample classified with confidence (middle) was calculated by summing the confidence scores (ranging from 0 to 1) for all classified reads and dividing by the total number of reads in the sample. Finally, the uncertain portion of a sample (right) was calculated by summing the complements of the confidence scores (1—score) for all classified reads and dividing by the total number of reads in the sample.

### Embodiment Examples

The following examples pertain to specific invention embodiments and point out specific features, elements, or steps that can be used or otherwise combined in achieving such embodiments.

In one embodiment there is provided a rare earth metal extracting bacterial consortium comprising: an acid secreting bacterium, a heavy metal resistant bacterium, an iron-sequestering molecule secreting bacterium; and a rare earth metal sequestering bacterium.

In one embodiment of a bacterial consortium, the acid secreting bacterium is an organic acid secreting bacterium.

In one embodiment of a bacterial consortium, the organic acid secreting bacterium is a citric acid secreting bacterium.

In one embodiment of a bacterial consortium, the citric acid secreting bacterium is Bacillus sp.

In one embodiment of a bacterial consortium, the organic acid secreting bacterium is a butyric acid secreting bacterium.

In one embodiment of a bacterial consortium, the butyric acid secreting bacterium is Butyrivibrio hungatei.

In one embodiment of a bacterial consortium, the organic acid secreting bacteria is an amino acid fermenting bacterium.

In one embodiment of a bacterial consortium, the amino acid fermenting bacterium is Clostridia venationis.

In one embodiment of a bacterial consortium, the heavy metal resistant bacterium resists heavy metal by active transport of metal ions, extracellular sequestration, intracellular sequestration, reduction of metal ions to insoluble metal, an extracellular barrier, or a combination thereof.

In one embodiment of a bacterial consortium, the heavy metal resistant bacterium is from the order Burkholderiales or genus Cupriavidus.

In one embodiment of a bacterial consortium, the heavy metal resistant bacterium grows in the presence of a heavy metal concentration of 30 grams per liter or more.

In one embodiment of a bacterial consortium, the heavy metal resistant bacterium contains a plasmid with at least 99% sequence identity to the pMOL30 plasmid of Burkholderiales.

In one embodiment of a bacterial consortium, the iron-sequestering molecule secreting bacterium secretes an iron-sequestering protein or an iron-sequestering siderophore.

In one embodiment of a bacterial consortium, the iron-sequestering molecule secreting bacterium is capable of or otherwise operable to secrete at least 10 grams of iron-sequestering protein per 1012 bacterial cells.

In one embodiment of a bacterial consortium, the iron-sequestering molecule secreting bacterium is Acinetobacter baumanni.

In one embodiment of a bacterial consortium, the rare earth metal sequestering bacterium sequesters rare earth metals by intracellular sequestration, extracellular sequestration, conversion to an insoluble metal, sequestration into a glycocalyx, sequestration by a specific binding protein, or a combination thereof.

In one embodiment of a bacterial consortium, the rare earth metal sequestering bacterium is capable of or otherwise operable to sequester at least 10 grams of rare earth metal per 1012 bacterial cells.

In one embodiment of a bacterial consortium, the rare earth metal sequestering bacterium is a xanthan gum secreting bacterium.

In one embodiment of a bacterial consortium, the xanthan gum secreting bacterium is Xanthomonas vesicatoria.

In one embodiment of a bacterial consortium, the rare earth metal sequestering bacterium is Peptostreptococcus anaerobius or a lactobacillus having a rare earth metal sequestering S-layer.

In one embodiment of a bacterial consortium, the consortium further comprises Collinsella aerofaciens.

In one embodiment of a bacterial consortium, the consortium further comprises axonopodis, brevis, anaerobius, frisia, coli, guillouiae, parainfluenzae, oryziterrae, Subtilis, or a combination thereof.

In one embodiment of a bacterial consortium, the acid secreting bacterium, the heavy metal resistant bacterium, the iron-sequestering molecule secreting bacterium, and the rare earth metal sequestering bacterium are aerobic bacteria.

In one embodiment of a bacterial consortium, the acid secreting bacterium, the heavy metal resistant bacterium, the iron-sequestering molecule secreting bacterium, and the rare earth metal sequestering bacterium are anaerobic bacteria.

In one embodiment of a bacterial consortium, a ratio of the number of acid secreting bacteria to heavy metal resistant bacteria to iron-sequestering molecule secreting bacteria to rare earth metal sequestering bacteria is 1-100 acid secreting bacteria to 1-100 heavy metal resistant bacteria to 1-100 iron-sequestering molecule secreting bacteria to 1-100 rare earth metal sequestering bacteria.

In one embodiment there is provided a composition (e.g. a bacterial consortium composition/growth composition) comprising a growth medium comprising water, magnesium sulfate, manganese chloride, cobalt chloride, calcium chloride, ammonium sulfate, soluble starch, and amino acids; and a bacterial consortium growing in the growth medium, the bacterial consortium comprising an acid secreting bacterium, a heavy metal resistant bacterium, an iron-sequestering molecule secreting bacterium, and a rare earth metal sequestering bacterium.

In one embodiment of a composition, the magnesium sulfate is 0.01 wt % to 1 wt % of the growth medium; manganese chloride is 0.01 wt % to 1 wt % of the growth medium; cobalt chloride is 0.01 wt % to 1 wt % of the growth medium; calcium chloride is 0.01 wt % to 1 wt % of the growth medium; ammonium sulfate is 0.01 wt % to 1 wt % of the growth medium; soluble starch is 0.01 wt % to 1 wt % of the growth medium; and amino acids are 0.01 wt % to 1 wt % of the growth medium.

In one embodiment there is provided a method of creating and/or growing a bacterial consortium as recited herein comprising providing a composition as recited herein, introducing bacterium as recited herein to the composition and maintaining the composition at a temperature from about 25° C. to about 50° C.

In one embodiment of a method of creating and/or growing a bacterial consortium, the method further comprises aerating the composition.

In one embodiment of a method of creating and/or growing a bacterial consortium, the method further comprises feeding the bacterial consortium with new growth medium at a rate from about 0.5 mL per 100 mL of the composition per hour to about 2 mL per 100 mL of the composition per hour.

It is to be understood that in any of the foregoing embodiments, or in the characterization or recitation of a bacterial consortium recited herein, that any or all of the individual bacterium of the consortium can be preselected in order to establish a consortium with specific properties, characteristics, or that provides specifically desired products, performance, etc. As such, in one embodiment, there is provided a rare earth metal extracting bacterial consortium comprising: a preselected acid secreting bacterium, a preselected heavy metal resistant bacterium, a preselected iron-sequestering molecule secreting bacterium; and a preselected rare earth metal sequestering bacterium. In another embodiment, there is provided a bacterial consortium that includes a mixture of preselected bacterium. Likewise, in some embodiments, a composition (e.g. a bacterial consortium composition/growth composition) can include a bacterial consortium comprising a preselected acid secreting bacterium, a preselected heavy metal resistant bacterium, a preselected iron-sequestering molecule secreting bacterium, and a preselected rare earth metal sequestering bacterium in the composition/growth composition.

Furthermore, in embodiments of a method of creating and/or growing a bacterial consortium as recited herein any or all (e.g. each) of the bacterium added to the composition (e.g. growth media) can be preselected bacterium as required in order to establish and grow the desired consortium.

While the forgoing examples are illustrative of the principles of invention embodiments in one or more particular applications, it will be apparent to those of ordinary skill in the art that numerous modifications in form, usage and details of implementation can be made without the exercise of inventive faculty, and without departing from the principles and concepts of the disclosure.

What is claimed is:

1. A rare earth metal extracting bacterial consortium comprising:
an organic acid secreting bacterium;
a heavy metal resistant bacterium that resists heavy metal by active transport of metal ions, extracellular sequestration, intracellular sequestration, reduction of metal ions to insoluble metal, an extracellular barrier, or a combination thereof;
an iron-sequestering molecule secreting bacterium that secretes an iron-sequestering protein or an iron-sequestering siderophore; and
a rare earth metal sequestering bacterium that sequesters rare earth metals by intracellular sequestration, extracellular sequestration, conversion to an insoluble metal, sequestration into a glycocalyx, sequestration by a specific binding protein, or a combination thereof.

2. The bacterial consortium of claim 1, wherein the organic acid secreting bacterium is a citric acid secreting bacterium.

3. The bacterial consortium of claim 2, wherein the citric acid secreting bacterium is Bacillus sp.

4. The bacterial consortium of claim 1, wherein the organic acid secreting bacterium is a butyric acid secreting bacterium.

5. The bacterial consortium of claim 4, wherein the butyric acid secreting bacterium is Butyrivibrio hungatei.

6. The bacterial consortium of claim 1, wherein the organic acid secreting bacteria is an amino acid fermenting bacterium.

7. The bacterial consortium of claim 6, wherein the amino acid fermenting bacterium is Clostridia venationis.

8. The bacterial consortium of claim 1, wherein the heavy metal resistant bacterium is from the order Burkholderiales or genus Cupriavidus.

9. The bacterial consortium of claim 1, wherein the heavy metal resistant bacterium grows in the presence of a heavy metal concentration of 30 grams per liter or more.

10. The bacterial consortium of claim 1, wherein the heavy metal resistant bacterium contains a plasmid with at least 99% sequence identity to the pMOL30 plasmid of Burkholderiales.

11. The bacterial consortium of claim 1, wherein the iron-sequestering molecule secreting bacterium secretes an iron-sequestering protein or an iron-sequestering siderophore.

12. The bacterial consortium of claim 1, wherein the iron-sequestering molecule secreting bacterium is capable of or otherwise operable to secrete at least 10 grams of iron-sequestering protein per 10¹² bacterial cells.

13. The bacterial consortium of claim 1, wherein the iron-sequestering molecule secreting bacterium is Acinetobacter baumanni.

14. The bacterial consortium of claim 1, wherein the rare earth metal sequestering bacterium is capable of or otherwise operable to sequester at least 10 grams of rare earth metal per 10¹² bacterial cells.

15. The bacterial consortium of claim 1, wherein the rare earth metal sequestering bacterium is a xanthan gum secreting bacterium.

16. The bacterial consortium of claim 15, wherein the xanthan gum secreting bacterium is Xanthomonas vesicatoria.

17. The bacterial consortium of claim 1, wherein the rare earth metal sequestering bacterium is Peptostreptococcus anaerobius or a lactobacillus having a rare earth metal sequestering S-layer.

18. The bacterial consortium of claim 1, further comprising Collinsella aerofaciens.

19. The bacterial consortium of claim 1, further comprising axonopodis, brevis, anaerobius, frisia, coli, guillouiae, parainfluenzae, oryziterrae, Subtilis, or a combination thereof.

20. The bacterial consortium of claim 1, wherein the acid secreting bacterium, the heavy metal resistant bacterium, the iron-sequestering molecule secreting bacterium, and the rare earth metal sequestering bacterium are aerobic bacteria.

21. The bacterial consortium of claim 1, wherein the acid secreting bacterium, the heavy metal resistant bacterium, the iron-sequestering molecule secreting bacterium, and the rare earth metal sequestering bacterium are anaerobic bacteria.

22. The bacterial consortium of claim 1, wherein a ratio of the number of acid secreting bacteria to heavy metal resistant bacteria to iron-sequestering molecule secreting bacteria to rare earth metal sequestering bacteria is 1-100 acid secreting bacteria to 1-100 heavy metal resistant bacteria to 1-100 iron-sequestering molecule secreting bacteria to 1-100 rare earth metal sequestering bacteria.
